(19) United States
(12) Patent Application Publication
(10) Pub. No.: US 2026/0060913 A1
(43) Pub. Date: Mar. 5, 2026

(54) BIOACTIVE WALNUT PEPTIDES FOR TREATING HAIR LOSS

(71) Applicant: L'OREAL, Paris (FR)

(72) Inventors: Eddy Hsi Chun WANG, Jersey City, NJ (US); Rebecca BARRESI, Robbinsville, NJ (US); I-chien LIAO, Princeton, NJ (US); Ying CHEN, Montville, NJ (US)

(73) Assignee: L'OREAL, Paris (FR)

(21) Appl. No.: 18/820,058

(22) Filed: Aug. 29, 2024

(51) Int. Cl. A61K 8/64; C07K 14/415

(52) U.S. Cl. CPC A61K 8/645; C07K 14/415

(57) ABSTRACT

Bioactive walnut peptides and use of the bioactive walnut peptides for topical application to the scalp are described. The bioactive walnut peptides may be incorporated into a pharmaceutical or cosmetic composition for application to the scalp. The pharmaceutical or cosmetic compositions and the one or more bioactive walnut peptides in the compositions are particularly useful for treating or preventing hair loss, and treating scalp conditions, such as dandruff, scalp psoriasis, and acne.

### REFERENCE TO THE SEQUENCE LISTING

The Sequence Listing submitted 29 Aug. 2024 as an XML file named “085137-OA24379_Sequence Listing,” created on 9 Aug. 2024 and having a size of 26 kilobytes is hereby incorporated by reference pursuant to 37 C.F.R. § 1.52(e)(5).

### FIELD OF THE DISCLOSURE

The present disclosure pertains to bioactive walnut peptides and methods for treating and preventing hair loss, and treating skin conditions of the scalp.

### BACKGROUND

Hair follicles, particularly in humans, are complex structures with distinct components, each made up of various specialized cells. Besides the cells responsible for hair shaft production and anchoring, most hair follicles also contain sebaceous glands, which produce sebum. Some follicles have apocrine glands, which are found in specific areas like the axilla. The structures within a hair follicle include the follicular papilla (FP) and the germinative epithelium (GE), collectively known as the bulb. The FP consists of mesenchymal cells and connective tissue, while the epithelial components include at least eight distinct cellular lineages: the outer root sheath (ORS), the companion layer (CL), the internal root sheath's Henle's layer (He), Huxley's layer (Hu), the cuticle of the internal root sheath (Csth), the cuticle of the hair shaft (Csft), the cortex of the hair shaft, and the medulla of the shaft (Med).

In humans, scalp hair and hair in certain other areas grow in follicular units. A typical follicular unit on the scalp consists of two to four terminal hair follicles, one or rarely two vellus hair follicles, along with their associated sebaceous glands, a neurovascular plexus, an erector pili muscle, and a circumferential band of adventitial collagen known as the “perifolliculum.”

Hair follicles are thought to produce about 20 individual hair shafts over their lifetime, cycling through phases of growth, shedding, involution, and new growth. While hair growth and follicle regeneration have been studied in mouse models, important differences exist between human and mouse hair follicle biology. In mice, a thick fur coat is vital for thermoregulation and other functions, with follicles predominantly producing terminal hair (fur). In contrast, human skin has areas covered with hair follicles that produce vellus hair, which is less visible. Mouse hair follicles have synchronous growth cycles early in life but become less synchronized with age, while human follicles progress through their growth cycles asynchronously. On an adult human scalp, approximately 80-90% of follicles are in the anagen (growth) phase, 10-20% are in telogen (resting), and 1-2% are in catagen (regression).

Hair follicle dermal papilla cells (DPCs) are crucial for hair growth, residing in the hair bulb and regulating the hair follicle growth cycle. Their ability to induce hair follicle formation, known as inductivity, is central to both hair loss prevention and hair growth promotion. DPCs secrete growth factors and signaling molecules that interact with epithelial cells in the hair follicle, driving cell proliferation and differentiation. This interaction is essential for initiating and sustaining hair growth, as DPCs play a pivotal role in the cyclical phases of hair growth: anagen (growth), catagen (regression), and telogen (rest). DPC activity influences the transitions between these phases, affecting hair length and thickness.

The shift from telogen to anagen is complex and involves several molecular signals, notably Wnt and Bone Morphogenetic Protein (BMP) signaling pathways. During telogen, Wnt signaling is inactive in the hair follicle. The transition to anagen is triggered by signals from DPCs, which activate Wnt signaling in epithelial stem cells located in the follicle's bulge region. This activation promotes the proliferation and differentiation of these stem cells, leading to the formation of a new hair shaft and the downward growth of the follicle into the dermis. In contrast, BMP signaling generally inhibits hair growth. Elevated BMP signaling during telogen helps maintain the follicle in its resting state. For anagen to commence, BMP signaling must be reduced, often involving inhibitory molecules that block BMP pathways and allow Wnt signaling to prevail, thus initiating hair growth.

Additionally, hypoxia (low oxygen levels) has been shown to enhance DPCs' inductivity in 2D models and promote angiogenesis, which is vital for healthy hair follicle and scalp development. A proper balance between these signaling pathways is essential for effective hair follicle development and cycling. Recent studies have also observed increased autophagy at the onset of anagen in the natural hair cycle, and activating autophagy in aged mice has been found to prevent (or slow) hair loss.

### SUMMARY OF THE DISCLOSURE

The instant disclosure relates to bioactive walnut peptides and use of the walnut peptides for topical application to the scalp for use in treating or preventing (or slowing) hair loss, and treating conditions of the scalp. The inventors surprisingly found bioactive walnut peptides positively influence physiological pathways associated with hair growth and the health of hair follicles, and downregulate physiological pathways determinantal to healthy hair growth and scalp functioning. For example, the bioactive walnut peptides stimulate or activate dermal papilla cells while down-regulating genes associated with cell cycle arrest and IL-1 (Interleukin-1) signaling, which is associated with inflammation, immune response, and apoptosis.

Walnuts are one of the most widely distributed and oldest nuts in the world. They have high nutritional value and are rich in oleic acid, linoleic acid, α-linolenic acid, and other unsaturated fatty acids, vitamins, and proteins. Walnuts are commonly used to make walnut oil because they contain a high lipid content. The residue remaining after lipid extraction is considered a by-product although it contains walnut protein and other useful components. Walnut protein is mainly composed of albumin, globulin, gliadin and glutenin.

Bioactive walnut peptides can be derived from walnut proteins or can be synthesized. For example, walnut peptides are obtained via enzymatic hydrolysis, fermentation hydrolysis, or chemical hydrolysis of walnut proteins or synthetically produced, for example, by solid-phase synthesis. Bioactive peptides that are particularly useful according to the instant disclosure typically have a molecular weight less than 6,000 Da and often less than 1,000 Da. Such walnut peptides can have from 2 to 50 amino acid residues but typically have from 2 or 3 amino acid residues up to about 20 amino acid residues.

Preferably, the bioactive walnut peptides have a minimum of 2 or 3 amino acid residues up to about 20 amino acid residues and impart a positive physiological or dermatological effect on the scalp, hair follicles, or both. Bioactive peptides include amino acids joined by covalent bonds, also referred to as amide or peptide bonds, whereas proteins are polypeptides with a greater molecular weight (MW), i.e., having more than 50 amino acid residues. Bioactive walnut peptides usually display hormone or drug-like activities and are classified based on their mode of action. Many bioactive peptides share some structural features for example, an amino acid residue length for 2 to 20 amino acids.

Useful bioactive walnut peptides according to the instant disclosure often include one or more amino acid residues selected from leucine, proline, or combinations thereof. In preferred embodiments, the one or more bioactive walnut peptides include from 2 to about 20 amino acids, wherein one or more of the amino acids is leucine, proline, or combinations thereof. Nonlimiting examples of bioactive walnut peptides having from 2 to about 20 amino acid residues, wherein one or more amino acid residues are leucine, proline, or combinations thereof comprises or consist of the peptides represented by SEQ ID NOS: 2-5, 7-9, and 11-46.

The one or more bioactive walnut peptides may have at least two amino acid residues (dipeptides) up to about 20 amino acid residues. Nonlimiting examples of dipeptides include the dipeptides represented by SEQ ID NOS: 1-12. The one or more bioactive walnut peptides may have at least three amino acid residues up to about 20 amino acid residues. Nonlimiting examples of bioactive walnut peptides having three amino acid residues include the peptides represented by SEQ ID NOS: 13-23. Further, the one or more bioactive walnut peptides may have at least four up to about 20 amino acid residues. Nonlimiting examples of bioactive walnut peptides having four amino acids include the peptides represented by SEQ ID NOS: 24-32. Even further, the one or more bioactive walnut peptides may include at least five amino acid residues up to about 20 amino acid residues. Nonlimiting examples of bioactive walnut peptides having five amino acid residues include the peptides represented by SEQ ID NOS: 33-39. In various embodiments, the one or more bioactive walnut peptides include at least six amino acid residues. Nonlimiting examples of bioactive walnut peptides having six amino acid residues include the peptides represented by SEQ ID NOS: 10-44. In yet further embodiments, the one or more bioactive walnut peptides include at least seven amino acid residues up to about 20 amino acid residues. Nonlimiting examples of bioactive walnut peptides having seven amino acid residues include the peptides represented by SEQ ID NOS: 45 and 46.

Particularly useful bioactive walnut peptides include peptides comprising or consisting of the peptides represented by SEQ ID NOS: 45 and 46, referred to throughout the instant disclosure as Peptide (I) or Walnut Peptide (I) (SEQ ID NO: 45) and Peptide (II) or Walnut Peptide (II) (SEQ ID NO: 46).

Peptide I
(SEQ ID NO: 45)
Thr-Trp-Leu-Pro-Leu-Pro-Arg (TWLPLPR),
and
Peptide II
(SEQ ID NO: 46)
Tyr-Val-Leu-Leu-Pro-Ser-Pro-Lys (YVLLPSPK).

As already mentioned, the bioactive walnut peptides are particularly useful for topical application to the scalp in methods for treating or preventing (or slowing) hair loss, and for treating conditions of the scalp. Accordingly, the instant disclosure is drawn to methods for treating the scalp, hair, or both. For example, the bioactive walnut peptides are useful in methods for preventing or slowing hair loss. With respect to conditions of the scalp, the bioactive walnut peptides are particularly useful in methods for preventing and treating dandruff, scalp psoriasis, acne, or combinations thereof. Typically, the one or more walnut peptides are applied to the scalp in a pharmaceutical or cosmetic composition, which includes a physiologically acceptable carrier. Common and useful physically acceptable carriers include water, water-soluble solvents, and mixtures thereof.

It can be beneficial to use more than one (two or more) bioactive walnut peptide in the methods and compositions describe throughout the disclosure. Different bioactive walnut peptides can function in concert to provide a variety of beneficial influences on the scalp and hair. For example, one or more bioactive walnut peptides may treat or prevent hair loss by potentiating secretion of growth factors and signaling molecules that interact with epithelial cells in the hair follicle, driving cell proliferation and differentiation. One or more other bioactive walnut peptides may combat or prevent chemical changes or natural effects of aging that can shorten the hair growth phase (anagen phase) leading to thinner hair and eventual hair loss. Due to their various mechanisms of action, two more bioactive walnut peptides can be used together to synergistically prevent hair loss and treat disorders of the scalp.

In various embodiments, use of two or more bioactive walnut peptides is preferred. The two or more bioactive walnut peptides may have similar activities or may provide different activities that benefit the hair and scalp. In further embodiments, use of three or more bioactive walnut peptides is preferred. The use of multiple bioactive peptides allows for the modification of more than one physiological mechanism in the treatment and prevention of hair loss. Combinations of bioactive walnut peptides can interact synergistically and provide benefits beyond the sum of the peptides' individual contributions. For example, the synergistic activity of a combination can be at least 5%, at least 10%, or at least 25% greater than the sum of the individual activities of the corresponding amounts of the bioactive walnut peptides.

The one or more bioactive walnut peptides are often incorporated into a pharmaceutical or cosmetic composition for application to the scalp. Pharmaceutical and cosmetic compositions typically include one or more bioactive walnut peptides and one or more physiologically acceptable carriers, for example water. Nonlimiting examples of physiologically acceptable carriers include water, water soluble solvents such as alcohols, polyols, and glycols, fatty compound such as oils, triglycerides, fatty acids, fatty alcohols, and the like. Pharmaceutical and cosmetic compositions include lotions, creams, serums, sprays, emulsions, gels, powders, dispersions, ointments, sticks, pastes, and foams.

The pharmaceutical or cosmetic compositions include a sufficient amount of the one or more bioactive walnut peptides to ensure a therapeutically effective amount of the one or more walnut peptides is administered to the scalp during use. Throughout the disclosure, when referencing application or administration to the scalp, it encompasses administration and application to the hair and hair follicles embedded within and extending from the scalp. Pharmaceutical and cosmetic composition include one or more bioactive walnut peptides and one or more physiologically acceptable carrier, for example water. Nonlimiting examples of physiologically acceptable carriers include water, water soluble solvents such as alcohols, polyols, and glycols, fatty compound such as oils, triglycerides, fatty acids, fatty alcohols, and the like. Pharmaceutical and cosmetic compositions include lotions, creams, serums, sprays, emulsions, gels, powders, dispersions, ointments, sticks, pastes, and foams.

It can be useful to combine one or more bioactive walnut peptides with one or more additional active agents, for example, active agents for treating or preventing hair loss, and treating conditions of the scalp. Accordingly, in various embodiments, the one or more bioactive walnut peptides are combined and/or administered together with one or more hair growth promoting agents or one or more active active agents for treating conditions of the scalp. Nonlimiting examples of hair growth promoting agents include androgen receptor inhibitors, androgen antagonists, and an antiandrogens. More specific but nonlimiting examples of hair growth promoting agents include episteride, finasteride, cyproterone acetate, alfatradiol, minoxidil, bimatoprost, bicalcutamide, spironolactone, flutamide, lantoanoprost, dutasteride, ketoconazole, tofacitinib, ruxolitinib, tacrolimus, bimatoprost, latanoprost, spironolactone, aldactone, kenalog-10, kenalog-40, triamcinolone, azulfidine, sulfasalazine, and sulfazine.

Nonlimiting examples of active agents for treating conditions of scalp include anti-dandruff agents, agents for treating psoriasis and/or inflammation of the scalp, anti-acne agents, and combinations thereof. Nonlimiting examples of anti-dandruff agents include ainz pyrithione, ketoxonazole, selenium sulfide, coal tar, and saliciylic acid. Nonlimiting examples of agents for treating psoriasis and/or inflammation include tacrolimuns, calcipotriene, corticosteroids, salicyclic acid, and coal tar. Nonlimiting examples of anti-acne agents include salicylic acid, benzoyl peroxide, alpha-hydroxy acids (AHAs), tea tree oil, retinoids, and ceramides.

### BRIEF DESCRIPTION OF THE DRAWINGS

Implementation of the present technology is described, by way of example only, with reference to the attached figures, wherein:

FIG. 1 is a circular plot showing relative gene scores for three bioactive walnut peptides (Walnut Peptides (I), (II), and (III)) tested as two different concentrations (100 μM and 250 μM); and

FIG. 2 is an illustration showing a hair follicle transitioning from the telogen phase to the anagen phase highlighting where different bioactive walnut peptides influence various physiological pathways of the anagen and telogen phases.

The various aspects of the disclosure are not limited to the results, arrangements, and representations shown in the drawings.

### DETAILED DESCRIPTION OF THE DISCLOSURE

The instant disclosure is drawn to bioactive walnut peptides and their topical use for treating and preventing hair loss, and treating disorders of the scalp. Bioactive walnut peptides include two to several dozen amino acids connected to one another through peptide bonds. Their molecular weight is generally less than 6000 Da, preferably less than 3,000 Da, and more preferably less than 1,000 Da. The term “peptide” in accordance with the present disclosure is a compound that includes an uninterrupted sequence of at least two amino acids within its structure and has a maximum of about 50 amino acid. The terms “di-peptide” or “dipeptide” as used herein refer to a compound that includes an uninterrupted sequence of two amino acids within its structure. The terms “tri-peptide” or “tripeptide” as used herein refer to a compound that includes an uninterrupted sequence of three amino acids within its structure. As used herein, a “tetra-peptide” or “tetrapeptide” is a compound that includes an uninterrupted sequence of four amino acids within its structure. These amino acids are indicated herein using a traditional one letter convention from left (N-terminal end) to right (C-terminal end). In this nomenclature, G is glycine, H is histidine, K is lysine, E is glutamic acid, and the like, according to well known and accepted nomenclature in the art.

A “bioactive” peptide for purposes of the instant disclosure has a minimum of 2 or 3 amino acid residues up to about 20 amino acid residues in length and has a measurable physiological effect on the scalp, hair follicles, and/or hair growth. Bioactive peptides include amino acids joined by covalent bonds, also referred to as amide or peptide bonds, whereas proteins are polypeptides with a greater molecular weight (MW) and typically more than 50 amino acid residues. Bioactive peptides usually display hormone or drug-like activities and are classified based on their mode of action. Many bioactive peptides share some structural features that include, for example, a peptide residue length of 2 to 20 amino acids.

The term “amino acid” as used herein includes and encompasses all naturally occurring amino acids, either in the D- or L-configuration if optically active, and the known non-native, synthetic, and modified amino acids, such as homocysteine, ornithine, norleucine, and p-valine. A list of non-natural amino acids may be found in The Peptides, Vol. 5 (1983), Academic Press, Chapter VI, by D. C. Roberts and F. Vellaccio, which is incorporated herein by reference in its entirety. The amino acids in the peptides of the present invention may be present in their natural L-configuration, unnatural D-configuration, or as a racemic mixture.

As used herein, the term “peptide” shall also refer to salts, deprotected forms, acylated forms of the peptide, deacylated forms of the peptide, enantiomers, diastereomers, racemates, prodrugs and hydrates of the above-mentioned peptide, unless otherwise specified. Diastereomers of the peptide are obtained when the stereochemical or chiral center of one or more amino acids is changed. The enantiomer has the opposite stereochemistry at all chiral centers. In various embodiments, the C-terminal of a peptide is synthesized as an amide to neutralize negative charge created by the C-terminal COOH. This modification can be added to help prevent enzyme degradation.

The term “prodrug” refers to any precursor compound which can generate or to release the above-mentioned peptide under physiological conditions. Such prodrugs are for instance larger peptides which are selectively cleaved to form the peptide of the invention. Further prodrugs are protected amino acids having protecting groups at the carboxylic acid and/or amino group. Suitable protecting groups for amino groups include, for example, the benzyloxycarbonyl, t-butyloxycarbonyl (BOC), formyl, and acetyl or acyl group. Suitable protecting groups for the carboxylic acid group are esters such as benzyl esters or t-butyl esters.

Useful bioactive walnut peptides according to the instant disclosure often include one or more amino acid residues selected from leucine, proline, or a combination thereof. In preferred embodiments, the one or more bioactive walnut peptides include from 2 to about 20 amino acids, wherein one or more of the amino acids is leucine, proline, or a combination thereof. Nonlimiting examples of bioactive walnut peptides having from 2 to about 20 amino acid residues, wherein at least one amino acid residue is leucine or proline include the peptides represented by SEQ ID NOS: 2-5, 7-9, and 11-46. The one or more bioactive walnut peptides comprising at least one leucine or proline residue have from 2 to about 15 amino acids, preferably from 2 to about 12 amino acids, more preferably from 2 to about 12 amino acids, and even more preferably from 2 to about 10 amino acids.

Nonlimiting examples bioactive walnut peptides having 2 amino acids (dipeptides) include the dipeptides represented by SEQ ID NOS: 1-12. Nonlimiting examples of bioactive walnut peptides having 2 amino acids (dipeptides) that have at least one leucine or proline residue include the dipeptides represented by SEQ ID NOS: 2-5 and 7-12. According, the one or more walnut peptides may be selected from peptides represented by SEQ ID NOS: 2-5 and 7-12. In further embodiments, the one or more bioactive walnut peptides are selected from peptides having one or more amino acid sequences represented by SEQ ID NOS: 2-5 and 7-12 and have from 3 to about 20 amino acids, from 3 to about 15 amino acids, from 3 to about 12 amino acids, from 3 to about 10 amino acids, from 3 to about 8 amino acids. In further embodiments, the one or more bioactive walnut peptides are selected from peptides having one or more amino acid sequences represented by SEQ ID NOS: 2-5 and 7-12 and have from 4 to about 20 amino acids, from 4 to about 15 amino acids, from 4 to about 12 amino acids, from 4 to about 10 amino acids, or from 4 to about 8 amino acids, from 5 to about 20 amino acids, from 5 to about 18 amino acids, from 5 to about 15 amino acids, from 5 to about 12 amino acids, from 5 to about 10 amino acids, from 5 to about 8 amino acids, from 6 to about 20 amino acids, from 6 to about 18 amino acids, from 6 to about 15 amino acids, from 6 to about 12 amino acids, or from 6 to about 10 amino acids.

In various embodiments, the one or more walnut peptides are selected from peptides having one more amino acid sequence represented by SEQ ID NOS: 1-12. In further embodiments, the one or more bioactive walnut peptides are selected from peptides having one or more amino acid sequences represented by SEQ ID NOS: 1-12 and have from 3 to about 20 amino acids, from 3 to about 15 amino acids, from 3 to about 12 amino acids, from 3 to about 10 amino acids, from 3 to about 8 amino acids. In further embodiments, the one or more bioactive walnut peptides are selected from peptides having one or more amino acid sequences represented by SEQ ID NOS: 1-12 and have from 4 to about 20 amino acids, from 4 to about 15 amino acids, from 4 to about 12 amino acids, from 4 to about 10 amino acids, or from 4 to about 8 amino acids, from 5 to about 20 amino acids, from 5 to about 18 amino acids, from 5 to about 15 amino acids, from 5 to about 12 amino acids, from 5 to about 10 amino acids, from 5 to about 8 amino acids, from 6 to about 20 amino acids, from 6 to about 18 amino acids, from 6 to about 15 amino acids, from 6 to about 12 amino acids, or from 6 to about 10 amino acids.

The one or more bioactive walnut peptides may include peptides having 3 amino acids. Nonlimiting examples of bioactive walnut peptides having 3 amino acids include the peptides represented by SEQ ID NOS: 13-23. Therefore, in various embodiments, the one or more walnut peptides may be selected from the peptides represented by SEQ ID NOS: 13-23. In further embodiments, the one or more bioactive walnut peptides are selected from peptides having one or more amino acid sequences represented by SEQ ID NOS: 13-23 and have from 3 to about 20 amino acids, from 3 to about 15 amino acids, from 3 to about 12 amino acids, from 3 to about 10 amino acids, from 3 to about 8 amino acids. In further embodiments, the one or more bioactive walnut peptides are selected from peptides having one or more amino acid sequences represented by SEQ ID NOS: 13-23 and have from 4 to about 20 amino acids, from 4 to about 15 amino acids, from 4 to about 12 amino acids, from 4 to about 10 amino acids, or from 4 to about 8 amino acids, from 5 to about 20 amino acids, from 5 to about 18 amino acids, from 5 to about 15 amino acids, from 5 to about 12 amino acids, from 5 to about 10 amino acids, from 5 to about 8 amino acids, from 6 to about 20 amino acids, from 6 to about 18 amino acids, from 6 to about 15 amino acids, from 6 to about 12 amino acids, or from 6 to about 10 amino acids.

The one or more bioactive walnut peptides may include peptides having 4 amino acids. Nonlimiting examples of bioactive walnut peptides having 4 amino acids include the peptides represented by SEQ ID NOS: 24-32. Therefore, in various embodiments, the one or more walnut peptides are selected from the peptides represented by SEQ ID NOS: 24-32. In further embodiments, the one or more bioactive walnut peptides are selected from peptides having one or more amino acid sequences represented by SEQ ID NOS: 24-32 and have from 4 to about 20 amino acids, from 4 to about 15 amino acids, from 4 to about 12 amino acids, from 4 to about 10 amino acids, from 4 to about 8 amino acids. In further embodiments, the one or more bioactive walnut peptides are selected from one or more amino acid sequences represented by SEQ ID NOS: 24-32 and have from 5 to about 20 amino acids, from 5 to about 18 amino acids, from 5 to about 15 amino acids, from 5 to about 12 amino acids, from 5 to about 10 amino acids, from 5 to about 8 amino acids, from 6 to about 20 amino acids, from 6 to about 18 amino acids, from 6 to about 15 amino acids, from 6 to about 12 amino acids, or from 6 to about 10 amino acids.

The one or more bioactive walnut peptides may include peptides having 5 amino acids. Nonlimiting examples of bioactive walnut peptides having 5 amino acids include the peptides represented by SEQ ID NOS: 33-39. Therefore, in various embodiments, the one or more walnut peptides are selected from the peptides represented by SEQ ID NOS: 33-39. In further embodiments, the one or more bioactive walnut peptides are selected from peptides having one or more amino acid sequence represented by SEQ ID NOS: 33-39 and have from 5 to about 20 amino acids, from 5 to about 15 amino acids, from 5 to about 12 amino acids, from 5 to about 10 amino acids, from 5 to about 8 amino acids. In further embodiments, the one or more bioactive walnut peptides are selected from peptides having one or more amino acid sequences represented by SEQ ID NOS: 33-39 and have from 6 to about 20 amino acids, from 6 to about 18 amino acids, from 6 to about 15 amino acids, from 6 to about 12 amino acids, or from 6 to about 10 amino acids.

The one or more bioactive walnut peptides may include peptides having 6 amino acids. Nonlimiting examples of bioactive walnut peptides having 6 amino acids include the peptides represented by SEQ ID NOS: 40-44. Therefore, in various embodiments, the one or more walnut peptides are selected from the peptides represented by SEQ ID NOS: 40-44. In further embodiments, the one or more bioactive walnut peptides are selected from peptides having one or more amino acid sequences represented by SEQ ID NOS: 40-44 and have from 6 to about 20 amino acids, from 6 to about 15 amino acids, from 6 to about 12 amino acids, from 6 to about 10 amino acids, from 6 to about 8 amino acids.

The one or more bioactive walnut peptides may include peptides having 7 amino acids. A nonlimiting example of a bioactive walnut peptide having 7 amino acids is the peptide represented by SEQ ID NO: 45. Therefore, in various embodiments, the one or more walnut peptides includes the peptide represented by SEQ ID NO: 45. In further embodiments, the one or more bioactive walnut peptides are selected from peptides having one or more amino acid sequences represented by SEQ ID NOS: 40-44 and have from 7 to about 20 amino acids, from 7 to about 15 amino acids, from 7 to about 12 amino acids, from 7 to about 10 amino acids, from 7 to about 8 amino acids.

The one or more bioactive walnut peptides include peptides having 8 amino acids. A nonlimiting example of a bioactive walnut peptide having 8 amino acids is the peptide represented by SEQ ID NO: 46. Therefore, in various embodiments, the one or more walnut peptides include the peptide represented by SEQ ID NO: 46. In further embodiments, the one or more bioactive walnut peptides are selected from peptides having the amino acid sequences represented by SEQ ID NO: 46 and have from 8 to about 20 amino acids, from 8 to about 15 amino acids, from 8 to about 12 amino acids, or from 8 to about 10 amino acids.

Particularly useful bioactive walnut peptides include peptides having the amino acid sequences represented by SEQ ID NOS: 45 and 46, referred to throughout the instant disclosure as Peptide (I) (or Walnut Peptide (I)) (SEQ ID NO: 45) and Peptide (II) (or Walnut Peptide (II)) (SEQ ID NO: 46).

Peptide I
(SEQ ID NO: 45)
Thr-Trp-Leu-Pro-Leu-Pro-Arg (TWLPLPR),
and
Peptide II
(SEQ ID NO: 46)
Tyr-Val-Leu-Leu-Pro-Ser-Pro-Lys (YVLLPSPK).

Accordingly, in a preferred embodiment, the one or more bioactive walnut peptides represented by SEQ ID NOS: 45 and 45 (Peptide (I) and Peptide (II)). In further embodiments, the one or more bioactive walnut peptides are selected from peptides having the amino acid sequences represented by SEQ ID NO: 45 and 46 and have from 7 to about 20 amino acids, from 7 to about 15 amino acids, from 7 to about 12 amino acids, from 7 to about 10 amino acids, from 7 to about 8 amino acids, 8 to about 20 amino acids, from 8 to about 15 amino acids, from 8 to about 12 amino acids, or from 8 to about 10 amino acids.

In a particularly preferred embodiment, the one or more bioactive walnut peptides include both the peptide represented by SEQ ID NO: 45 and the peptide represented by SEQ ID NO: 46.

In further embodiments, the one or more bioactive walnut peptides may be selected from peptides of Formula (I) and Formula (II), shown below.

(I)
X-Thr-Trp-Leu-Pro-Leu-Pro-Arg-Z
(II)
X-Tyr-Val-Leu-Leu-Pro-Ser-Pro-Lys-Z,

wherein,
X represents the primary amine function of the N-terminal amino acid, free or substituted by a protecting group selected from an acetyl group, a benzoyl group, a tosyl group, or a benzyloxycarbonyl group, and
Z represents the hydroxyl group of the carboxyl function of the C-terminal amino acid, free or substituted by a protecting group selected from a C₁-C₂₀, NH₂, NHY, or NYY, wherein Y represents a C1-C4 alkyl.

Bioactive walnut peptides can be derived by hydrolysis of walnut proteins into small molecular peptides with molecular weights between the molecular weight of individual amino acids and the molecular weights of the proteins. This can be carried out using biological or chemical methods. For example, bioactive peptides can be prepared by enzymatic procedures, fermentation, and with chemical methods.

Bioactive walnut peptides can be synthesized by coupling the carboxyl group or C-terminus of one amino acid to the amino group or N-terminus of another. Due to the possibility of unintended reactions, protecting groups are sometimes necessary. Chemical peptide synthesis starts at the C-terminal end of the peptide and ends at the N-terminus. Peptides can be synthesized either by solid-phase peptide synthesis, by liquid-phase peptide synthesis, or by fragment condensation. In principle, the seemingly simple formation of a peptide bond can be accomplished using all the procedures available in organic chemistry for the synthesis of carboxylic acid amides.

The general process for synthesizing peptides on solid-phase (e.g., resin) starts by attaching the first amino acid, the C-terminal residue, to the resin. To prevent the polymerization of the amino acid, the alpha amino group and the reactive side chains are protected with a temporary protecting group. Once the amino acid is attached to the resin, the resin is filtered and washed to remove byproducts and excess reagents. Next, the N-alpha protecting group is removed in a deprotection process, and the resin is again washed to remove byproducts and excess reagents. Then the next amino acid is coupled to the attached amino acid. This is followed by another washing procedure, which leaves the resin-peptide ready for the next coupling cycle. The cycle is repeated until the peptide sequence is complete. Then typically, all the protecting groups are removed, and the peptide resin is washed, and the peptide is cleaved from the resin.

Enzymatic hydrolysis involves using commercial enzymes to obtain bioactive peptides. The enzymes are responsible for cleaving the peptide bonds established in the protein, thereby releasing the encrypted peptide. For the enzyme to carry out its activities, it is important for the enzyme bind the substrate and continue with enzymatic catalysis. The enzyme has specific active sites containing residues that form temporary bonds with the substrate and residues that catalyze the reaction with the substrate. In this way, binding sites and catalytic sites are formed, respectively. The bonds forming the enzyme-substrate complex are usually hydrogen bonds, hydrophobic bonds, or Van der Waals interactions. Enzymatic hydrolysis can generally be carried out in three ways: (i) under traditional batch conditions; (ii) using immobilized enzymes; or (iii) using ultrafiltration membranes. Numerous proteolytic enzyme are known and include those described, for example, in Cruz-Casas et al., Enzymatic Hydrolysis and Microbial Fermentation: The Most Favorable Biotechnological Methods for the Release of Bioactive Peptides (FOOD CHEM (OXF). 3:100047, Dec. 30, 2021), which is incorporated herein by reference in its entirety.

Microbial fermentation is a biotechnological process through which bioactive peptides can be obtained. This process involves using microorganisms capable of producing proteolytic enzymes with the objective that these enzymes hydrolyze proteins into shorter peptides. The microorganisms generally used are bacteria, fungi, or yeasts, which may be present in the substrate indigenously or added as a starter culture. The microbial fermentation process can be divided into several systems. However, submerged fermentation and solid-state fermentation are the most widely used.

Submerged fermentation uses a culture of microorganisms in a liquid medium, which contains nutrients. This system is suitable for microorganisms with high water level activities, such as bacteria, and offers the advantage that the generated bioactive peptides are easy to purify. Solid-state fermentation uses microbial growth on nutrient-rich solid substrates. It has the advantage of releasing nutrients in a controlled way and is suitable for fungi and microorganisms with fewer moisture requirements.

Bioactive walnut peptides may be produced using any method known to those skilled in the art such as those disclosed in Merrifield, R. B., Solid Phase Peptide Synthesis I., J. AM. CHEM. SOC. 85:2149-2154 (1963); Carpino, L. A. et al., [(9-Fluorenylmethyl)Oxy] Carbonyl (Fmoc) Amino Acid Chlorides: Synthesis, Characterization, And Application To The Rapid Synthesis Of Short Peptides, J. ORG. CHEM. 37:51:3732-3734; Merrifield, R. B. et al., Instrument For Automated Synthesis Of Peptides, ANAL. CHEM. 38:1905-1914 (1966); or Kent, S. B. H. et al., High Yield Chemical Synthesis Of Biologically Active Peptides On An Automated Peptide Synthesizer Of Novel Design, IN: PEPTIDES 1984 (Ragnarsson U., ed.) Almqvist and Wiksell Int., Stockholm (Sweden), pp. 185-188, which are all incorporated herein by reference in their entirety.

### Pharmaceutical or Cosmetic Compositions

The one or more bioactive walnut peptides are typically incorporated into a pharmaceutical or cosmetic composition for application to the scalp. Pharmaceutical and cosmetic compositions include one or more bioactive walnut peptides and one or more physiologically acceptable carriers. Nonlimiting examples of physiologically acceptable carriers include water, water soluble solvents such as alcohols, polyols, and glycols, fatty compound such as oils, triglycerides, fatty acids, fatty alcohols, and the like.

The pharmaceutical or cosmetic composition includes a sufficient amount of the one or more bioactive walnut peptides to ensure a therapeutically effective amount of the one or more walnut peptides is administered to the scalp during use. The term “therapeutically effective amount” as used herein refers to an amount sufficient to treat or prevent hair loss, or treat a scalp condition. Throughout the disclosure, when referencing application or administration to the scalp, it encompasses topical administration and application to the hair and hair follicles embedded within and extending from the scalp. In various embodiments, the pharmaceutical or cosmetic composition includes from about 0.001 to about 10 wt. % of one or more bioactive walnut peptides. In further embodiments, the pharmaceutical or cosmetic compositions include from about 0.001 to about 8 wt. %, about 0.001 to about 5 wt. %, about 0.001 to about 3 wt. %, about 0.001 to about 2 wt. %, about 0.001 to about 1 wt. %, about 0.001 to about 0.5 wt. %, about 0.01 to about 8 wt. %, about 0.01 to about 5 wt. %, about 0.01 to about 3 wt. %, about 0.01 to about 2 wt. %, about 0.01 to about 1 wt. %, about 0.01 to about 0.5 wt. %, about 0.1 to about 8 wt. %, about 0.1 to about 5 wt. %, about 0.1 to about 3 wt. %, about 0.1 to about 2 wt. %, about 0.1 to about 1 wt. %, about 0.1 to about 0.5 wt. % of the one or more bioactive walnut peptides. Preferably, the pharmaceutical or cosmetic composition includes from about 0.05 to about 8 wt. %, more preferably about 0.1 to about 5 wt. %, and even more preferably about 0.1 to about 3 wt. % of the one or more bioactive walnut peptides.

The therapeutically effective amount of the one or more bioactive walnut peptides will vary depending on the bioactive walnut peptide and the combination of bioactive walnut peptides. In various embodiments, the therapeutically effective amount is from about 1 μg to about 50 mg (50,000 μg) per cm² of skin of the scalp. In further embodiments, the therapeutically effective amount of the one or more walnut peptides is from about 1 μg to about 40 mg, about 1 μg to about 30 mg, about 1 to about 20 mg, about 1 μg to about 10 mg, about 1 μg to about 8,000 μg, about 1 μg to about 5,000 μg, about 1 μg to about 2,000 μg, about 1 μg to about 1,000 μg, about 10 μg to about 40 mg, about 10 μg to about 30 mg, about 10 to about 20 mg, about 10 μg to about 10 mg, about 10 μg to about 8,000 μg, about 10 μg to about 5,000 μg, about 10 μg to about 2,000 μg, about 10 μg to about 1,000 μg, about 100 μg to about 50 mg, about 100 μg to about 40 mg, about 100 μg to about 30 mg, about 100 μg to about 20 mg, about 100 μg to about 10 mg, about 100 μg to about 8,000 μg, about 100 μg to about 5,000 μg, about 100 μg to about 2,000 μg, about 100 μg to about 1,000 μg, about 500 μg to about 50 mg, about 500 μg to about 40 mg, about 500 μg to about 30 mg, about 500 μg to about 30 mg, about 500 μg to about 20 mg, about 500 μg to about 10 mg, about 500 to about 8,000 μg, about 500 μg to about 5,000 μg, about 500 μg to about 2,000 μg, or about 500 μg to about 1,000 μg per cm² of skin of the scalp.

In preferred embodiment, the pharmaceutical or cosmetic composition includes two or more bioactive walnut peptides. In further embodiments, the pharmaceutical or cosmetic compositions includes two or more bioactive walnut peptides provided that at least one of the bioactive walnut peptides comprises or consists of the amino acid sequence represented by SEQ ID NO: 45. In another embodiment, the pharmaceutical or cosmetic compositions includes two or more bioactive walnut peptides provided that at least one of the bioactive walnut peptides comprises or consists of the amino acid sequence represented by SEQ ID NO: 46. In a preferred embodiment, the pharmaceutical or cosmetic composition include both a peptide comprising or consisting of the amino acid sequence represented by SEQ ID NO: 45 and a peptide comprising or consisting of the amino acid sequence represented by SEQ ID NO: 46, preferably the peptide represented by SEQ ID NO: 45 and the peptide represented by SEQ ID NO: 46 (i.e., Peptides (I) and (II)).

For pharmaceutical or cosmetic compositions that include Peptide (I), the amount of Peptide (I) in the compositions will vary but is typically in an amount from about 0.01 to about 8 wt. %, based on the total weight of the pharmaceutical or cosmetic composition. Preferably, the pharmaceutical or cosmetic composition comprises from about 0.01 to about 5 wt. %, about 0.01 to about 3 wt. %, about 0.01 to about 2 wt. %, about 0.01 to about 1 wt. %, about 0.01 to about 0.5 wt. %, about 0.1 to about 8 wt. %, about 0.1 to about 5 wt. %, about 0.1 to about 3 wt. %, about 0.1 to about 2 wt. %, about 0.1 to about 1 wt. %, or about 0.1 to about 0.5 wt. % of Peptide (I), based on the total weight of the pharmaceutical or cosmetic composition.

For pharmaceutical or cosmetic compositions that include Peptide (II), the amount of Peptide (II) in the compositions will vary but is typically in an amount from about 0.01 to about 8 wt. %, based on the total weight of the pharmaceutical or cosmetic composition. Preferably, the pharmaceutical or cosmetic composition comprises from about 0.01 to about 5 wt. %, about 0.01 to about 3 wt. %, about 0.01 to about 2 wt. %, about 0.01 to about 1 wt. %, about 0.01 to about 0.5 wt. %, about 0.1 to about 8 wt. %, about 0.1 to about 5 wt. %, about 0.1 to about 3 wt. %, about 0.1 to about 2 wt. %, about 0.1 to about 1 wt. %, or about 0.1 to about 0.5 wt. % of Peptide (II), based on the total weight of the pharmaceutical or cosmetic composition.

Peptide (I) and Peptide (II) may be in a weight ratio of about 1:10 to about 10:1. Preferably, Peptide (I) and Peptide (II) are in a weight ratio of about 1:5 to about 5:1, more preferably about 1:2 to about 2:1, and even more preferably about 1:1.

Two or more bioactive walnut peptides will often function to treat or prevent hair loss, or treat scalp conditions in a synergistic manner. For example, the two or more bioactive walnut peptides can synergistically prevent (or slow) hair loss, and treat conditions of the scalp. For example, the two or more bioactive walnut peptides can synergistically reduce occurrences and/or severity of dandruff, scalp psoriasis, and acne. “Synergism,” as used herein, is when two or more substances or factors work together in such a way that their combined effect is greater than the sum of their individual effects. In other words, their interaction produces a result that is more effective or beneficial than if each worked alone. For example, the synergistic activity or result based on a combination of two or more bioactive walnut peptides can be at least 5%, at least 10%, or at least 25% greater than the sum of the peptide's individual contribution.

The pharmaceutical or cosmetic compositions include one or more bioactive walnut peptides and a physiologically acceptable carrier. A “physiological carrier” as used herein is a carrier that is appropriate and safe for application to the scalp of a human. A particularly common physiologically acceptable carriers is water. However, physiologically acceptable carriers can be oil, fats, organic solvents, and the like, provided they are appropriate and safe for application to the scalp. Nonlimiting examples of physiologically acceptable carriers include water, water-soluble solvents such as alcohols, polyols, and glycols, fatty compound such as oils, triglycerides, fatty acids, fatty alcohols, petrolatum, and the like. The pharmaceutical and cosmetic compositions of the instant disclosure can be lotions, creams, serums, sprays, emulsions, gels, powders, dispersions, ointments, sticks, pastes, or foams.

Pharmaceutical and cosmetic compositions including the one or more bioactive walnut peptides can be formulated in various forms, for example, lotions, creams, serums, sprays, emulsions, gels, powders, dispersions, ointments, sticks, pastes, and foams. In addition to a physiologically acceptable carrier, the pharmaceutical and cosmetic composition may optionally include one or more of the following:
Emollients
Suspending agents,
Emulsifying agents, and
Thickeners.

### Emollients

One or more emollients may also optionally be included in the pharmaceutical or cosmetic composition described herein. An emollient generally refers to an ingredient that can help maintain a soft, smooth, and supple skin appearance. Emollients generally remain on the skin surface or in the stratum corneum and act as a moisturizer or lubricant and reduce delamination. Nonlimiting examples of emollients include acetylarginine, acetylated lanolin, algal extract, polyethylene glycol-6 esters from apricot kernel oil, polyethylene glycol-11 esters from avocado oil, bis-polyethylene glycol-4 dimethicone, butoxyethyl stearate, glycol esters, alkyl glycol ethers esters, cetyl laurate, coconut polyethylene glycol-10 esters, alkyl tartrates, diethyl sebacate, dihydrocholesteryl butyrate, dimethiconol, dimyristyl tartrate, distearare-5 lauroylg utamat, etilavokadat, ethylhexyl myristate, glyceryl isostearate, glyceryl oleate, geksildetsilstearat, geksilizostearat, hydrogenated palm glycerides, hydrogenated soy glycerides, hydrogenated glycerides of fat izostearilneopentanoat, isostearyl palmitate, izotridetsilizononanoat, laureth-2 acetate, lauryl polyglyceryl-6 cetearyl glycol ether, metilglyutset-20 benzoate, mineral oil, palm oil, coconut oil, miret-3 palmitate, octyldecanol, octyldodecanol, Odontella aurita oil, 2-oleamido-1,3 octadecandiol, pal commercial glycerides, glycerides of polyethylene glycols avocado, polyethylene glycol castor oil, copolymer of polyethylene glycol-2/dodecyl glycol, glycerides of polyethylene glycol shea butter, phytol, raffinose, stearyl citrate, glycerides of sunflower seed oil, non-ointment, small tocopheryl glucoside.

### Suspending Agents

The pharmaceutical or cosmetic composition of the present invention may optionally include one or more suspending agents, preferably in a concentration effective to suspend the water-insoluble material in a dispersed form in the compositions or to modify the viscosity of the composition. Such concentrations will vary. Nonetheless, in certain embodiments, the pharmaceutical and cosmetic composition includes from about 0.1 to about 10 wt. %, more preferably from about 0.25% to about 5.0, or even more preferably from about 0.5 to about 3 wt. % of the one or more suspending agents, based on the total weight of the composition. Nonlimiting examples include vinyl polymers, such as cross-linked acrylic acid polymers, called carbomer, cellulose derivatives and modified cellulose polymers such as methyl cellulose, ethyl cellulose, nitrocellulose, carboxymethyl cellulose, crystalline cellulose, cellulose powder, polyvinylpyrrolidone, polyvinyl alcohol, guar gum, hydroxypropyl guar gum, gum Arabic, galactan, locust bean gum, pectin, agar, starch (rice, corn, potato, wheat), algal colloids (algae extract), microbiological polymers such as dextran, succinoglycan, pullulan, starch-based polymers such as carboxymethyl starch, methyl starch, alginic acid polymers such as sodium alginate, alginic acid propylene glycol esters, acrylate polymers such as sodium polyacrylate, polyacrylate, polyacrylamide, polyethyleneimine and inorganic minutes water soluble material such as bentonite, aluminum magnesium silicate, laponite, hectonite, and anhydrous silicic acid.

Other optional suspending agents include crystalline suspending agents that can be resolved into acyl derivatives, long chain amine oxides, long chain acyl derivatives, and mixtures thereof. Said preferred suspending agents include fatty acid ethylene glycol esters, fatty acid alkanolamides, long chain fatty acid esters (for example, stearyl stearate, cetyl palmitate, etc.); long chain esters of long chain alkanolamides (for example, stearamide diethanolamide distearate, stearamide monoethanol amide stearate); and glyceryl esters (e.g., glyceryl distearate, trihydroxystearin, tribhengen). Other suitable suspending agents include primary amines containing a fatty alkyl fragment containing at least about 16 carbon atoms, examples of which include palmitamine or stearamine, and secondary amines containing two fatty alkyl fragments, each of which contains at least about 12 carbon atoms examples of which include dipalmitoylamine or di (hydrogenated fat) amine. Other suitable suspending agents include phthalic acid di amide (hydrogenated fat) and a crosslinked maleic anhydride/methyl vinyl ether copolymer.

### Emulsifying Agents

Nonlimiting examples of emulsifying agents include condensation products of alkylene oxides with fatty acids (ie alkylene oxide fatty acid esters), condensation products of alkylene oxides with 2 moles of fatty acids (ie fatty acid alkylene oxide diesters), condensation products alkylene oxides with fatty alcohols (ie fatty alcohol alkylene oxide esters), condensation products of alkylene oxides with both fatty acids and fatty alcohols [i.e. where a portion of the polyalkylene oxide is esterified at one end with a fatty acid and esterified (ie, via an ether bond) at the other end with a fatty alcohol]. Non-limiting examples of non-ionic surfactants derived from said alkylene oxide include cetet-6, cetet-10, cetet-12, cetetaret-6, cetetaret-10, cetetaret-12, stearet-6, stearet-10, stearet-12, stearet-21, PEG-6 stearate, PEG-10 stearate, PEG-100 stearate, PEG-12 stearate, PEG-20 glyceryl stearate, PEG-80 glyceryl tallowat, PEG-10 glyceryl stearate, PEG-30 glyceryl cocoate, PEG-80 glyceryl coco, PEG-200 glyceryl tallowat, PEG-8 dilaurate, PEG-10 distearate and mixtures thereof. Other applicable non-ionic surfactants include polyhydroxyamide fatty acid surfactants. A particularly preferred surfactant corresponding to the above structure is N-methylglucoside coconut alkylamide. Preferred among nonionic surfactants are surfactants selected from the group consisting of stearet-21, ceteareth-20, ceteareth-12, sucrose cocoate, stearet-100, PEG-100 stearate and mixtures thereof. Other non-ionic surfactants suitable for use in this application include sugar esters and polyesters, alkoxylated sugar esters and polyesters, C1-C30 fatty acid esters C1-C30 fatty alcohols, alkoxylated C1-C30 ester derivatives C1-C30 fatty alcohol fatty acids, alkoxylated C1-C30 fatty alcohol esters, polyglyceryl C1-C30 fatty acid esters, C1-C30 polyol esters, C1-C30 polyol esters, alkyl phosphates, polyoxyalkylene fatty ether forfates, fatty acid amides, acylactylates and mixtures thereof. Non-limiting examples of these emulsifiers include: polyethylene glycol 20 sorbitan monolaurate (polysorbate 20), polyethylene glycol 5 soy sterol, stearet-20, cetearet-20, PPG-2 methyl glucose ether distearate, cetet-10, polysorbate 80, cetyl phosphate, cetyl phosphate, cetyl phosphate, cetyl phosphate, cetyl phosphate, polysorbate 60, glyceryl stearate, polyoxyethylene 20 sorbitan triolcat (polysorbate 85), sorbitan monolaurate, polyoxyethylene 4 lauryl ether sodium stearate, polyglyceryl-4 isostearate, hexyl laurate, PPG-2 methyl glucose ether distearate, PEG-100 and their PEG-100. Another group of non-ionic surfactants useful herein is a mixture of fatty acid esters based on a mixture of sorbitan or a sorbitol fatty acid ester and a sucrose fatty acid ester, where the fatty acid in each example is preferably C8-C24, more preferably C10-C20 fatty acid.

The one or more emulsifying agents are in an amount sufficient to emulsify the pharmaceutical or cosmetic compositions. For example, the one or more emulsifying agents may be in an amount from about 0.1 to about 10 wt. %, based on the total weight of the pharmaceutical or cosmetic composition. In further embodiments, the one or more emulsifying agents may be in an amount of about 0.1 to about 8 wt. %, about 0.1 to about 5 wt. %, about 0.1 to about 3 wt. %, about 0.1 to about 2 wt. %, about 0.5 to about 10 wt. %, about 0.5 to about 8 wt. %, about 0.5 to about 5 wt. %, about 0.5 to about 2 wt. %, or about 0.5 to about 1 wt. %, based on the total weight of the pharmaceutical or cosmetic composition.

### Thickeners

Thickeners suitable for inclusion in the pharmaceutical or cosmetic composition described herein. Nonlimiting examples include acrylamide copolymer, agarose, amylopectin, bentonite, calcium alginate, calcium carboxymethyl cellulose, carbomer, carboxymethylchitin, cellulose gum, dextrin, gelatin, hydrogenated hydroxymethyl hydroxy cellulose hydroxypropyl, hydroxyethyl hydroxypropyl hydroxypropyl, hydroxyethyl hydroxypropyl, hydroxyethyl hydroxypropyl, hydroxyethyl hydroxypropyl, hydroxyethyl hydroxypropyl, hydroxyethyl hydroxypropyl, hydroxypropyl, hydroxypropyl, hydroxypropyl, hydroxypropyl, hydroxypropyl; magnesium alginate, methyl cellulose, microcrystalline cellulose, pectin, various polyethylene glycols, polyacrylic acid, polymethacrylic acid, polyvinyl alcohol, various sexes ipropylene glycols, copolymers of sodium acrylates, sodium carrageenan, xanthan gum and/or yeast beta-glucan, or mixtures thereof.

More generally, carboxylic acid polymers are useful thickeners. Polymers of carboxylic acids are crosslinked compounds containing one or more monomers derived from acrylic acid, substituted acrylic acids and salts and esters of said acrylic acids and substituted acrylic acids, wherein the crosslinking agent contains two or more carbon-carbon double bonds and comes from polyhydric alcohol. Examples of commercially available carboxylic acid polymers useful herein include carbomers, which are homopolymers of acrylic acid crosslinked with sucrose or pentaerythrotol allyl ethers. Carbomers are available as Carbopol® 900 Series from B.F. Goodrich (e.g. Carbopol® 954). In addition, other suitable carboxylic acid based polymeric agents include copolymers of C10-30 alkyl acrylates with one or more monomers of acrylic acid, methacrylic acid or esters of one of its short chains (i.e., C1-4 alcohol), the crosslinking agent is a sucrose or pentaerythritol allyl ether. These copolymers are known as acrylate/C10-30 alkyl acrylate crosspolymers and are commercially available as B.F. Carbopol® 1342, Carbopol® 1382, Pemulen TR-1 and Pemulen TR-2. Goodrich. Examples of preferred thickeners based on carboxylic acid polymers useful in this application include thickeners selected from carbomers, acrylate/C10-30 alkyl acrylate cross-polymers, or mixtures thereof.

Moreover, according to certain embodiments, the thickeners are selected from polysaccharides. Nonlimiting examples of polysaccharide thickeners include cellulose, carboxymethyl hydroxyethyl cellulose, cellulose acetate propionate carboxylate, hydroxyethyl cellulose, hydroxyethyl cellulose, hydroxypropyl cellulose, hydroxypropyl methyl cellulose, sodium methyl hydroxyethyl cellulose hydroxyethyl cellulose and hydroxyethyl methyl hydroxyethyl hydroxyethyl cellulose hydroxyethyl methyl hydroxyethyl hydroxyethyl cellulose hydroxyethyl hydroxyethyl cellulose hydroxyethyl methyl hydroxyethyl hydroxyethyl. Alkyl-substituted celluloses are also useful. In these polymers, the hydroxy groups of the cellulosic polymer are hydroxylated (preferably hydroxyethylated or hydroxypropylated) to form hydroxylated cellulose, which is then further modified with a C10-30 straight or branched chain alkyl group via an ether bond. Typically, these polymers are straight chain or branched chain esters of C10-30 alcohols and hydroxyalkyl celluloses. Examples of alkyl groups applicable in this application include groups selected from stearyl, isostearyl, lauryl, myristyl, cetyl, isocetyl, cocoyl (e.g., an alkyl group derived from coconut oil alcohols), palmityl, oleyl, linoleil, linolenyl, recinoleil, behenyl and mixtures thereof. A preferred alkyl hydroxyalkyl cellulose ester is a material called cetyl hydroxyethyl cellulose, which is an ester of cetyl alcohol and hydroxyethyl cellulose, in accordance with the Perfume and Cosmetics and Perfume Association (CTFA). The indicated material is sold under the trade name Natrosol® CS Plus from Aqualon Corporation (Wilmington, Delaware). Further examples can be found in The International Cosmetic Ingredient Dictionary and Handbook, the Cosmetic Bench Reference—Directory of Cosmetic Ingredients, offered by the United States Pharmacopeia (USP) and National Formulary (NF), and other references to cosmetic and pharmaceutical ingredients known in the art. technicians. Other useful polysaccharides include scleroglucans, which are a straight chain (1-3) linked glucose units (1-6), where every three glucose units are linked, a commercially available example of which is Clearogel™ CS11 from Michel Mercier Products Inc. (Mountainside, New Jersey).

Other useful thickeners include those derived from natural sources. Nonlimiting examples include gum arabic, agar, algin, alginic acid, ammonium alginate, amylopectin, calcium alginate, calcium carrageenan, carnitine, carrageenan, dextrin, gelatin, gellan gum, guar gum hydrochloride, gidrohydrochloride, gidrohydrochloride, gidrohydrochloride, hydrochloride hyaluronic acid, hydrated silicon dioxide, hydroxypropylchitosan, hydroxypropyl gum, karaya gum, kelp, fruit tree resin, natto gum, potassium alginate, potassium carrageenan, propylene glycol alginate, sclerotium gum, sodium carboxymethyl dextran, dextran, sodium carrageenan, tragacanth gum, xanthan gum, or mixtures thereof.

In addition, the compositions may optionally contain polyacrylamide polymers, in particular nonionic polyacrylamide polymers, including substituted branched or unbranched polymers. Other polyacrylamide polymers useful herein include multi-block copolymers of acrylamides and substituted acrylamides with acrylic acids and substituted acrylic acids.

The one or more thickener are in an amount sufficient to thicken the pharmaceutical or cosmetic compositions. For example, the one or more thickeners may be in an amount from about 0.05 to about 5 wt. %, based on the total weight of the pharmaceutical or cosmetic composition. In further embodiments, the one or more thickeners may be in an amount of about 0.05 to about 3 wt. %, about 0.05 to about 2 wt. %, about 0.05 to about 1 wt. %, about 0.1 to about 5 wt. %, about 0.1 to about 3 wt. %, about 0.1 to about 2 wt. %, about 0.1 to about 1 wt. %, about 0.5 to about 5 wt. %, about 0.5 to about 3 wt. %, or about 0.5 to about 2 wt. %, based on the total weight of the pharmaceutical or cosmetic composition.

### Additional Active Agents

It can be useful to combine one or more bioactive walnut peptides with one or more additional active agents for treating or preventing hair loss, and treating conditions of the scalp. For example, additional active agents include hair growth promoting agents and active agents for treating conditions of the scalp. Nonlimiting examples of hair growth promoting agents include androgen receptor inhibitors, androgen antagonists, and an antiandrogens. More specific but nonlimiting examples of hair growth promoting agents include episteride, finasteride, cyproterone acetate, alfatradiol, minoxidil, bimatoprost, bicalcutamide, spironolactone, flutamide, lantoanoprost, dutasteride, ketoconazole, tofacitinib, ruxolitinib, tacrolimus, bimatoprost, latanoprost, spironolactone, aldactone, kenalog-10, kenalog-40, triamcinolone, azulfidine, sulfasalazine, and sulfazine.

Nonlimiting examples of active agents for treating conditions of scalp include anti-dandruff agents, agents for treating psoriasis and/or inflammation of the scalp, anti-acne agents, and combinations thereof. Nonlimiting examples of anti-dandruff agents include zinc pyrithione, ketoxonazole, selenium sulfide, coal tar, and saliciylic acid. Nonlimiting examples of agents for treating psoriasis and/or inflammation include tacrolimuns, calcipotriene, corticosteroids, salicyclic acid, and coal tar. Nonlimiting examples of anti-acne agents include salicylic acid, benzoyl peroxide, alph-hydroxy acids (AHAs), tea tree oil, retinoids, and ceramides.

In a preferred embodiment, one of the one or more additional active agents is biotin.

In a preferred embodiment, one of the one or more additional active agents is baicalin or Scutellaria baicalensis extract.

In a preferred embodiment, one or more additional active agents is a botanical extract. Nonlimiting examples of botanical extracts that are useful for treating or prevent hair loss include rosemary extract, peppermint extract, saw palmetto extract, ginseng extract, nettle extract, aloe vera extract, green tea extract bamboo extract horsetail extract, fenugreek extract, grapeseed extracts (and/or proanthoxyanidins), skullcap, and eclipta.

In a preferred embodiment, the pharmaceutical or cosmetic composition includes one or more additional active agents selected from botanicals and botanical extracts of Eclipta alba, Polygonum multiforum, Pisum sativum, Momordica charanita, Astragalus membranaceus, Sophora flavescens, Angelica sinensis, Caffeine, Malus domestica, Vitis viniferis, Rosmarinus officinalis, Polygonum multiflorum, Eclipta prostrat, Punica granatum, Scutellaria baicalensis, Ganoderma lucidum, Ocimum basilicum, Moringa olefiera, Chenopodium quinoa, and Lupine protein. The one or more additional active agent of the instant disclosure preferably includes those set forth in US20240075089, which is incorporated herein by reference in its entirety.

A list of preferred botanicals that are useful as additional active agents is provided below.

| | Approx- | | |
|---|---|---|---|
| | imate | | |
| | Extract | Preferred | Preferred |
| Botanical | Ratio | Standardization | Constituents |
| Chenopodium | | | |
| quinoa | | | |
| Eclipta prostrata | 10 | 5% lactones as | Tannins, saponins, |
| | | wedelolactone | nicotine, ecliptine |
| Lupinus | | | |
| Malus domestica | 30 | 50% Polyphenols | Polyphenols, |
| | | by UV | pectin, potassium |
| Momordica | 10 | 5% bitter | Charantin, |
| charantia | | principles by UV | momordicosides, |
| | | | momordicine |
| Moringa olifiera | | | |
| Ocimum basilicum | | | |
| Pisum sativum | | | |
| Rosmarinus | | | |
| officinalis | | | |
| Scutellaria | 12 | 30% flavones as | Flavones (baicalin, |
| baicalensis | | baicalin by UV | baicalein, |
| | | | wogonin)sterols |
| Vitis viniferis | 10-15 | >=95% | 5-15% Catechin |
| | | Proanthocyanadins | Monomers, >=80% |
| | | as Catechins by | Procyanidolic |
| | | HPLC | Oligomers |

A list of preferred additional active agents are provided below with preferred minimum and maximum concentrations (weight percent) for use in the pharmaceutical or cosmetic compositions of the instant case.

| Ingredients | Min | Max |
|---|---|---|
| Biotin | 0.005% | 1.00% |
| Caffeine | 0.0001% | 1.00% |
| Chenopodium quinoa | 0.1% | 2.00% |
| Eclipta prostrata | 0.01% | 4.00% |
| Lupinus | 0.01% | 6.00% |
| Malus domestica | 0.01% | 4.00% |
| Methylsulfonylmethane | 0.005% | 2.00% |
| Momordica charantia | 0.01% | 4.00% |
| Moringa olifiera | 0.01% | 6.00% |
| Ocimum basilicum | 0.01% | 6.00% |
| Pisum sativum | 0.01% | 6.00% |
| Rosmarinus officinalis | 0.01% | 4.00% |
| Scutellaria baicalensis | 0.01% | 4.00% |
| Vitamin E (Alpha | 0.005% | 2.00% |
| tocopherol) | | |
| Vitis viniferis | 0.01% | 4.00% |

In a preferred embodiment, the pharmaceutical or cosmetic composition includes one or more additional active agents selected from Eclipta prostrata, Scutellaria baicalensis, and proanthocyanidin, and optionally any one or a combination of Malus domestica, Rosmarinus officinalis, Moringa olifiera, and biotin.

In a preferred embodiment, one or more additional active agents are selected from proanthocyanidins, which includes procyanidin, procyanadin, anthocyanidin, anthocyanadin, anthocyanin, celphinidin, cyanidin, delphinidin, malvidin, pelargonidin, peonidin, petunidin. Proanthocyanidins are in a group of compounds called polyphenols and belong to a subclass of compounds called flavonoids that can be found in many plants, including; apples, pine bark, cinnamon, aronia fruit, cocoa beans, grape seed, grape skin, bilberry, cranberry, black currant, green tea, black tea, cocoa beans, Quercus petraea and Q. robur heartwood, agaf palm, field beans (Vicia faba), gallipoli rose, lingonberry, cranberry, black elderberry, chokeberry, black currant, blueberry, strawberry, persimmon, banana, carob bean, Chinese quince, chokeberries, rose hips, medlar, mulberry, plum, apricot, walnut, silverberry, pomegranate, triticale, sorghum, red cabbage, birch and Ginkgo biloba.

In various embodiments, the total amount of additional active agents in the pharmaceutical or cosmetic composition, other than the one or more bioactive walnut peptides, are in an amount greater than zero to about 9 wt. %, greater than zero to about 8 wt. %, greater than zero to about 7 wt. %, greater than zero to about 6 wt. %, greater than zero to about 5 wt. %, greater than zero to about 4 wt. %, greater than zero to about 3 wt. %, greater than zero to about 2 wt. %; about 10 ppm to about 10 wt. % (100,000 ppm), about 10 ppm to about 5 wt. % (50,000 ppm), about 10 ppm to about 2.5 wt. % (25,000 ppm), about 10 ppm to about 1 wt. % (10,000 ppm), about 10 ppm to about 0.5 wt. % (5,000 ppm), about 10 ppm to about 0.3 wt. % (3,000 ppm), about 10 ppm to about 0.2 wt. % (2,000 ppm), about 10 ppm to about 0.1 wt. % (1,000 ppm), about 10 ppm to 500 ppm; about 0.1 to about 10 wt. %, about 0.1 to about 5 wt. %, about 0.1 to about 2.5 wt. %, about 0.1 to about 1 wt. %, about 0.1 to about 0.5 wt. %; about 1 to about 10 wt. %, about 1 to about 8 wt. %, about 1 to about 6 wt. %, about 1 to about 5 wt. %, about 1 to about 4 wt. %, about 1 to about 3 wt. %; about 2 to about 10 wt. %, about 2 to about 8 wt. %, about 2 to about 6 wt. %, about 2 to about 5 wt. %, about 2 to about 4 wt. %; about 3 to about 10 wt. %, about 3 to about 8 wt. %, about 3 to about 6 wt. %, about 3 to about 5 wt. %; about 4 to about 10 wt. %, about 4 to about 8 wt. %, or about 4 to about 6 wt. %, based on the total weight of the pharmaceutical or cosmetic composition.

### Optional Exclusions

Bioactive walnut peptides can be derived from walnut proteins or can be synthesized. For example, walnut peptides are obtained via enzymatic hydrolysis, fermentation hydrolysis, or chemical hydrolysis of walnut proteins or synthetically produced, for example, by solid-phase synthesis. In various embodiments, the one or more bioactive walnut peptides are isolated from other constituents of the walnut and walnut tree from which the bioactive walnut peptide is derived. Walnuts and walnut trees include constituents such as fats, proteins, fiber, vitamins, minerals, phytosterols, etc. The term “walnut tree” in the context of the instant disclosure includes all parts of the walnut tree, such as the roots, trunk, bark, limbs, leaves, hulls, and husks. Accordingly, the one or more walnut peptides useful in the methods and compositions disclosed herein may be isolated from other constituents of the walnut or the walnut tree. For example, the one or more walnut peptide may be free or essentially free from walnut fats, walnut peptides and proteins (other than the one or more bioactive walnut peptides specified), walnut fibers, and the like. Further pharmaceutical or cosmetic composition comprising one or more walnut peptides may be free or essentially free from walnut fats, walnut peptides and proteins (other than the one or more bioactive walnut peptides specified), walnut fibers, and the like.

Walnuts, particularly their hulls, are useful for producing a variety of dye compounds. The primary dye compounds in walnuts and walnut trees is juglone and tannins. Therefore, the one or more bioactive walnut peptides may be isolated from the dye compounds of the walnut and walnut tree, including juglone and tannins. Similarly, the pharmaceutical or cosmetic compositions comprising the one or more bioactive walnut peptides preferably is free or essentially free from dye compounds of the walnut and walnut tree. For example, in various embodiments, the pharmaceutical or cosmetic composition comprises no dye compounds from walnuts and walnut trees or comprises less than 1 wt. %, preferably less than 0.1 wt. %, more preferably less than 0.01 wt. %, more preferably less than 0.001 wt. %, even more preferably less than 0.0001 wt. %, and even more preferably less than 0.00001 wt. %, based on the total weight of the pharmaceutical or cosmetic composition.

In a preferred embodiment, the bioactive walnut peptides are isolated from juglone, a naturally occurring compound found primarily in the hulls and leaves of walnut trees, particularly the black walnut (Juglans nigra). Juglone is a type of naphthoquinone. The bioactive walnut peptides are preferably isolated from walnut naphthoquinones including juglone. The pharmaceutical or cosmetic compositions comprising the one or more bioactive walnut peptides are preferably free or essentially free from juglone. For example, in various embodiments, the pharmaceutical or cosmetic composition comprises no juglone or comprises less than 1 wt. %, preferably less than 0.1 wt. %, more preferably less than 0.01 wt. %, more preferably less than 0.001 wt. %, even more preferably less than 0.0001 wt. %, and even more preferably less than 0.00001 wt. %, based on the total weight of the pharmaceutical or cosmetic composition.

Furthermore, the pharmaceutical or cosmetic compositions comprising the one or more bioactive walnut peptides may be free or essentially free from walnut naphthoquinones, including juglone. For example, in various embodiments, the pharmaceutical or cosmetic composition comprises no walnut naphthoquinones or comprises less than 1 wt. %, preferably less than 0.1 wt. %, more preferably less than 0.01 wt. %, more preferably less than 0.001 wt. %, even more preferably less than 0.0001 wt. %, and even more preferably less than 0.00001 wt. %, based on the total weight of the pharmaceutical or cosmetic composition.

In a preferred embodiment, the bioactive walnut peptides are isolated from tannins. Tannins are polyphenolic compounds found in many plants and are known for their astringent properties. In walnuts, tannins are mainly present in the skin or hull of the nut. These compounds contribute to the bitter taste of walnut skins and have antioxidant and antimicrobial properties. While they are more concentrated in the walnut hull and leaf, some tannins are also found in the edible part of the nut, although in lower amounts. In a preferred embodiment, the pharmaceutical or cosmetic composition comprising the one or more bioactive walnut peptides are free or essentially free from tannins. For example, in various embodiments, the pharmaceutical or cosmetic composition comprises no walnut tannins or comprises less than 1 wt. %, preferably less than 0.1 wt. %, more preferably less than 0.01 wt. %, more preferably less than 0.001 wt. %, even more preferably less than 0.0001 wt. %, and even more preferably less than 0.00001 wt. %, based on the total weight of the pharmaceutical or cosmetic composition.

In various embodiments, the one or more bioactive walnut peptides do not include the peptide represented by SEQ ID NO: 47 (also referred to throughout the disclosure as Peptide (III) or Walnut Peptide (III).

Peptide (III)
(SEQ ID NO: 47)
KVPPLLY (Lys-Val-Pro-Pro-Leu-Leu-Tyr)

For example, the one or more bioactive walnut peptides may comprise less than 1 wt. % of Peptide (III) based on the total weight of the one or more bioactive walnut peptides. More preferably, the one or more bioactive walnut peptides include less than 0.1 wt. %, more preferably less than 0.01 wt. %, more preferably less than 0.001 wt. %, even more preferably less than 0.0001 wt. %, and even more preferably less than 0.00001 wt. % of Peptide (III), based on the total weight of the one or more bioactive walnut peptides. Similarly, pharmaceutical or cosmetic compositions comprising the one or more bioactive walnut peptides may be free or essentially free from Peptide (III). For example, the pharmaceutical or cosmetic compositions comprising the one or more bioactive walnut peptides may include less than 1 wt. % of Peptide (III), based on the total weight of the pharmaceutical or cosmetic composition. Preferably, the pharmaceutical or cosmetic composition includes less than 0.01 wt. %, more preferably less than 0.001 wt. %, even more preferably less than 0.0001 wt. %, and even more preferably less than 0.00001 wt. % of Peptide (III), based on the total weight of the pharmaceutical or cosmetic composition.

### Methods of Treatment

As already mentioned, the bioactive walnut peptides are particularly useful for topical application to the scalp in methods for treating or preventing hair loss, and for treating conditions of the scalp. More specifically, the bioactive walnut peptides are useful in methods for increasing hair density, increasing follicular density, increasing hair shaft thickness, increasing hair length, preventing hair loss, reducing hair loss, or any combination thereof, or example, in an individual in need thereof. The methods include applying an effective amount of one or more bioactive walnut peptides to the scalp of an individual, preferably an individual in need thereof. In a further embodiment, the individual in need thereof is an individual with one or more disorders selected from alopecia greata, androgenic alopecia, alopecia areata, alopecia universalis, involutional alopecia, trichotillomania, telogen effluvium, anagen effluvium, cicatricial, alopecia, scarring alopecia, scalp thinning, hair shaft abnormalities, infectious hair disorders, genetic disorders, and hair loss due to chemotherapy, hormonal imbalance, fungal infection, medication intake, chemical hair treatment, or aging.

In a preferred embodiment, the bioactive walnut peptides are useful in methods for treating alopecies in an individual in need thereof, wherein the individual in need thereof is an individual suffering from adrenergic alopecia, telogen effluvium, alopecia areata, traumatic alopecia, anagen effluvium, nutritional deficiencies, metabolic defects, marked weight loss, diabetes, hypervitaminosis, hypovitaminosis, zinc deficiency, alopecia vulgaris, alopecia pustulosa, alopecia erythrodermica, alopecia arthropathica, para-alopecia, palmoplantar pustulosis, ichthyoses, keratodermias, and genodermatoses with pathological cornification disorders. The methods include applying an effective amount of one or more bioactive walnut peptides to the scalp of an individual in need thereof.

In further embodiments, the one or more bioactive walnut peptides are useful in methods for stimulating or activating dermal papilla cells of the scalp and/or hair follicles, potentiating Fibroblast Growth Factor (FGF) and/or Vascular Endothelial Growth Factor (VEGF) in the scalp and/or hair follicles, protecting or preserving hair follicles by regulating or managing oxidative stress (managing Reactive Oxygen Species (ROS)) in the scalp and/or hair follicles, down-regulating cell cycle arrest and/or IL-1 (Interleukin-1) signaling in hair follicles and/or in the scalp, treating inflammation of the hair follicles and/or the scalp, or combinations thereof.

In a preferred embodiment, the one or more bioactive walnut peptides are useful in methods for treating conditions of the scalp. Nonlimiting examples of conditions of the scalp include dandruff, scalp psoriasis, and acne.

For example, the bioactive walnut peptides are useful in methods for treating, preventing, or slowing hair loss. With respect to conditions of the scalp, the bioactive walnut peptides are particularly useful in methods for preventing and treating dandruff, scalp psoriasis, acne, or combinations thereof. Typically, the one or more walnut peptides are applied to the scalp in a pharmaceutical or cosmetic composition, which includes a physiologically acceptable carrier. Common and useful physically acceptable carriers include water, water-soluble solvents, and mixtures thereof.

It can be beneficial to use more than one (two or more) bioactive walnut peptide in the methods and compositions describe throughout the disclosure. Different bioactive walnut peptides can function in concert to provide a variety of beneficial influences on the scalp and hair. For example, one or more bioactive walnut peptides may treat hair loss by potentiating secretion of growth factors and signaling molecules that interact with epithelial cells in the hair follicle, driving cell proliferation and differentiation. One or more other bioactive walnut peptides may combat or prevent chemical changes or natural effects of aging that can shorten the hair growth phase (anagen phase) leading to thinner hair and eventual hair loss. Due to their various mechanisms of action, two more bioactive walnut peptides can be used together to synergistically treat, prevent (or slow) hair loss, and treat disorders of the scalp.

In various embodiments, use of two or more bioactive walnut peptides is preferred. The two or more bioactive walnut peptides may have similar activities or may provide different activities that treat or prevent hair loss, and treat conditions of the scalp. In further embodiments, use of three or more bioactive walnut peptides is preferred. The use of multiple bioactive peptides allows for the modification of more than one physiological mechanism in the treatment of skin. Combinations of bioactive walnut peptides can interact synergistically and provide benefits beyond the sum of the peptides' individual contributions. For example, the synergistic activity of a combination can be at least 5%, at least 10%, or at least 25% greater than the sum of the individual activities of the corresponding amounts of the bioactive walnut peptides.

Various changes can be made in the above-described compositions and methods without departing from the scope of the invention. Accordingly, it is intended that all disclosure contained in the above description and in the examples given below, shall be interpreted as illustrative and not in a limiting sense.

### EXAMPLES

### Example 1

### Dermal Papilla Cell (DPC) Inductivity

Dermal papilla cell (DPC) inductivity refers to a state where DPCs retain the ability to be stimulated or activated to promote the development of hair follicles. It has been shown that growth factors, like Fibroblast Growth Factor (FGF) and Vascular Endothelial Growth Factor (VEGF), can stimulate DPCs and enhance their inductive properties, promoting hair growth (Ito et al., Decapeptide with fibroblast growth factor (FGF)-5 partial sequence inhibits hair growth suppressing activity of FGF-5. J CELL PHYSIO., 2003 November; 197(2):272-83).

DPCs were cultured in follicle dermal papilla cell basal medium (from Promocell product number C-26501) that was supplemented with 4% fetal calf serum, (FCS), 0.004 ml/ml of bovine pituitary extract, 5 μg/ml of insulin, and 1 nf/ml of fibroblast growth factor (FGF). The dermal papilla cells were treated with the bioactive walnut peptides for 24 hours using 100 μM 250 μM concentrations of each peptide. At the end of the culture, RNA extraction and bulk RNA sequencing was subsequently carried out. RNA was extracted from each sample following standard protocols, quantified, and subjected to high-throughput RNAseq via DNA Nanoballs technology.

Bioinformatic analysis of the transcriptomic data was performed with gene ontology-driven and gene panel-driven approaches (Examples 3 and 4). In gene ontology (GO) term analysis, all genes significantly modulated were analyzed to see which pre-defined pathways they tend to fall into (an unsupervised approach) (Example 3). In the gene panel approach, DP cell specific marker genes were selected and segregate into biological functions of interest. Then a relative gene-panel score based on the integration of p-value and fold change was calculated for each panel of genes compared to the negative control (a curated approach) (Example 4).

### Example 2

### Autophagy Inductivity

Hair growth requires a significant amount of the body's energy. During the active growth phase of hair follicles (Anagen), the metabolic demand is highest because hair cells are rapidly dividing and synthesizing new proteins. Alpha-Ketoglutarate (α-KG) is a key metabolite in cellular metabolism and plays an important role in autophagy, which is the process by which cells degrade and recycle their own components. It has effects on cellular metabolism, oxidative stress, and signaling pathways, which are applicable to healthy hair growth. A-KG exhibits antioxidant properties that regulate oxidative stress in cells. Reducing oxidative stress can protect hair follicle cells from damage and support their longevity and overall health. In addition, by managing Reactive Oxygen Species (ROS), helps prevent damage to hair follicle cells. Alpha-KG supports hair growth through its roles in metabolism, oxidative stress management, signaling pathways, and stem cell regulation. Its impact on these processes can contribute to healthier hair follicles and potentially improve hair growth.

Bioactive walnut peptides can be studied on mice, for example, C57BL/6 mice, or genetically modified strains that exhibit hair loss with compositions containing one or more bioactive walnut peptides, such as the composition set forth in Example 5. Various concentrations of the one or more bioactive walnut peptides can be tested. Minoxidil, a vasodilator used to treat pattern hair loss can be used as a positive control (Messenger and Rundegren, Minoxidil: Mechanisms of Action on Hair Growth, BR. J. DERMATOL. 150:186-194 (2004)).

Mice are divided into two groups: a treatment group and a control group. The treatment group will receive once daily or twice daily topical applications of the peptide compositions, while the control group will receive a vehicle (placebo). The dorsal skin of the mice is shaved or depilated to facilitate observation of hair growth. The peptide compositions are applied once or twice daily for a specified period, for example, four weeks or eight weeks. The mice are monitored throughout the treatment period for overall health and signs of hair growth. Changes in hair density, color, and texture are also noted.

At the end of the treatment period, the mice are euthanize following ethical guidelines and skin samples collected from both the treated and control areas for analysis. For histological and biochemical analysis, the collected skin samples are processed by embedding them in paraffin, sectioned, and stained for histological examination. Immunohistochemistry or immunofluorescence techniques are used to detect autophagy markers, such as LC3 and p62, in the skin tissues. Additionally, Western blotting on tissue extracts can be carried out to quantify levels of autophagy-related proteins and quantitative PCR used to assess gene expression related to autophagy.

The bioactive walnut peptides can be evaluated for autophagy inductivity in mice as outlined, for example, in Chai et al., Stimulation of Hair Growth by Small Molecules that Activate Autophagy, CELL REPORTS, 25:3143-3421 (2019) and Sun et al., Autophagy Induces Hair Follicle Stem Cell Activation and Hair Follicle Regeneration by Regulating Glycolysis, CELL AND BIOSCIENCE, 14(6) (2024), which are incorporated herein by reference in their entirety.

### Example 3

### Gene Ontology-Driven Analysis

Gene Ontology (GO)-driven analysis is a method used in bioinformatics and computational biology to interpret and organize large sets of gene or protein data based on the Gene Ontology. It provides a structured and standardized mechanism to describe gene functions, processes, and cellular locations. To ascertain what, if any, biological processes are influenced by Walnut Peptide (I) (SEQ ID NO: 45), Walnut Peptide (II) (SEQ ID NO: 46), and Walnut Peptide (III) (SEQ ID NO: 47), an initial analysis of transcriptomic data was performed with GO-driven analysis, in which all genes that are significantly modulated are analysis to see whether they impact any pre-defined biological pathways. All genes that were statistically significantly upregulated or downregulated were analyzed for their association to biological processes. The multivariate results are presented as the ratio of differentially expressed genes in the dataset that matches the genes in public databases for any given biological process (rich ratio) as well as the significance of such association (Q value).

The literature-based panels used in the GO-driven analysis are shown below.

Literature Based Panels

| WNT | BMP | FGF | SHH |
|---|---|---|---|
| NR5A2 | BMP6 | FGFR1 | PTCH1 |
| FZD2 | NOG | SPRY4 | PTCH2 |
| WNT5A | BMP2 | FGF5 | GLI1 |
| SOX11 | GREM11 | FGF1 | GLIS2 |
| FRZB | BMP4 | FGF7 | DYRK1B |
| FZD6 | BMP4 | FGF14 | |
| NDP | BMP7 | FGF20 | |
| DKK1 | FST | PIK3R1 | |
| JUN | BMP5 | SPRY1 | |
| WIF1 | SOSTDC1 | MAPK12 | |
| SOX8 | JUN | FGFR3 | |
| SOX9 | PITX2 | FGF18 | |
| SOX2 | | | |
| TLE4 | | | |
| BAMBI | | | |
| LEF1 | | | |

### Walnut Peptide (I)

Walnut Peptide (I) (SEQ ID NO: 45) was tested on DP cells at concentrations of 100 μM and 250 μM and found to influence biological pathways implicated in hair growth. In particular, Walnut Peptide (I) (SEQ ID NO: 45) showed a significant up-regulation of genes involved in hair follicle development, angiogenesis, and hypoxia. Moreover, Walnut Peptide (I) (SEQ ID NO: 45) also significantly down-regulated genes associated with cell cycle arrest and IL-1 (Interleukin-1) signaling, which is associated with inflammation, immune response, and apoptosis. The data therefore suggests that Walnut Peptide (I) (SEQ ID NO: 45) has a positive influence on biological pathways that treats or prevents hair loss, while simultaneously downregulating biological pathways that are detrimental to hair growth.

### Walnut Peptide (II)

Walnut Peptide (II) (SEQ ID NO: 46) was tested on DP cells at concentrations of 100 μM and 250 μM and found to influence biological pathways implicated in hair growth. Walnut Peptide (II) (SEQ ID NO: 46) was found to positively influence biological pathways associated with mitochondrial activity and adenosine triphosphate (ATP) production. Surprisingly, Walnut Peptide (II) (SEQ ID NO: 46) downregulated biological pathways that are detrimental to hair growth, for example, it down-regulated inflammatory pathways.

### Walnut Peptide (III)

Walnut Peptide (III) (SEQ ID NO: 47) was tested on DP cells at concentrations of 100 μM and 250 μM and not found to influence biological pathways implicated in hair growth. Unlike Walnut Peptide (I) and Walnut Peptide (II), Walnut Peptide (III) did not show benefits associated with treating or preventing hair loss.

In summary, for the genes that were downregulated by Walnut Peptide (I), the pathways associated were more diverse. Notably, cell cycle arrest, IL1 signaling, IL4 response, and apoptosis regulation were downregulated, suggesting that Walnut Peptide (I) reduces the negative signals causing adverse effects on the activity of DPCs, potentially creating a healthier microenvironment. With Walnut Peptide (II) (for both concentrations), a different set of pathways were associated with the upregulated genes. Most of these genes were associated with mitochondrial and ATP/energy production pathways. Suggesting that Walnut Peptide (II) has a different mechanism of action compared to Walnut Peptide (I) and acts on powering the DPCs to provide the energy needed for rapid growth. For the genes that were downregulated by Walnut Peptide (II), the associated pathways were similar to Walnut Peptide (I) where there was a strong association with antigen-presentation (could trigger inflammation) and cell cycle arrest. In addition, the genes responsible for negative regulation of ATP production were also downregulated with Walnut Peptide (II).

With Walnut Peptide (III), there were very few significant associations observed for both upregulated and downregulated genes. Genes associated with ATP production and response to hypoxia were found with the lower concentration of Walnut Peptide (III), but overall the effects were weaker compared to Walnut Peptides (I) and (II).

### Example 4

### Gene Panel-Driven Analysis

In addition to the GO-driven analysis described above, a Gene Panel (GP)-driven analysis was carried out. A GP-driven analysis is used like a GO-driven analysis in genomics and clinical research to study specific sets of genes related to particular diseases, conditions, or biological processes. The GP-driven approach, however, involves analyzing a predefined panel of genes associated with a specific condition or trait of interest. It is a type of “curated” approach. Genes are selected based on their known or suspected association with specific conditions, their role in relevant biological pathways, or their potential impact on biological outcomes. Often, panels are customized for a specific research project or clinical need.

The GP-driven analysis allows for a targeted analysis using panels of genes that belongs to pathways of interest that may not be found in the publica domain. Genes that are specific to DPCs (determined from published literatures) were isolated to determine the exact changes in DPC transcriptomics. Importantly, a panel of genes relevant to the inductivity of DPCs were evaluated, as well as autophagy and hypoxia genes. The aggregated score of from each of the genes (whether it is positive or negative) were used to determine a relative effect against the negative control. The unique panels used for the testing are shown below.

Unique Panels

| Inductivity | Autophagy | Hypoxia |
|---|---|---|
| LRP4 | ATG9A | SLC2A1 |
| RGS2 | ULK1 | VEGFA |
| TRPS1 | WIPI1 | HIF1A |
| WNT5A | ULK2 | CA9 |
| ALPL | BCL2 | EGLN3 |
| SEMA4C | PINK1 | BNIP3 |
| NOG | NBR1 | |
| NDO | WDR45 | |
| RBP1 | SQSTM1 | |
| MEF2C | ATG16L1 | |
| SOX2 | GABARAP | |
| SPRY4 | ATG3 | |
| BMP4 | ATG14 | |
| BAMBI | MAP1LC3B | |
| LEF1 | GABARAPL1 | |
| | ATG10 | |
| | OPTN | |
| | UVRAG | |

With Walnut Peptide (I), a relative increase of inductivity and hypoxia was readily observed, while autophagy and WNT signaling were observed to a lesser extent. These results are consistent with the gene ontology (GO)-driven analysis (Example 3) while providing additional insight into other positive regulators for DPCs relevant to anagen entry. Surprisingly, strong downregulation of BMP signaling was observed, which is known to work against WNT signaling. Taken together, in the targeted analysis, Walnut Peptide (I) induces the major positive signals and reduces the negative signals for DPC activity relative to the negative control.

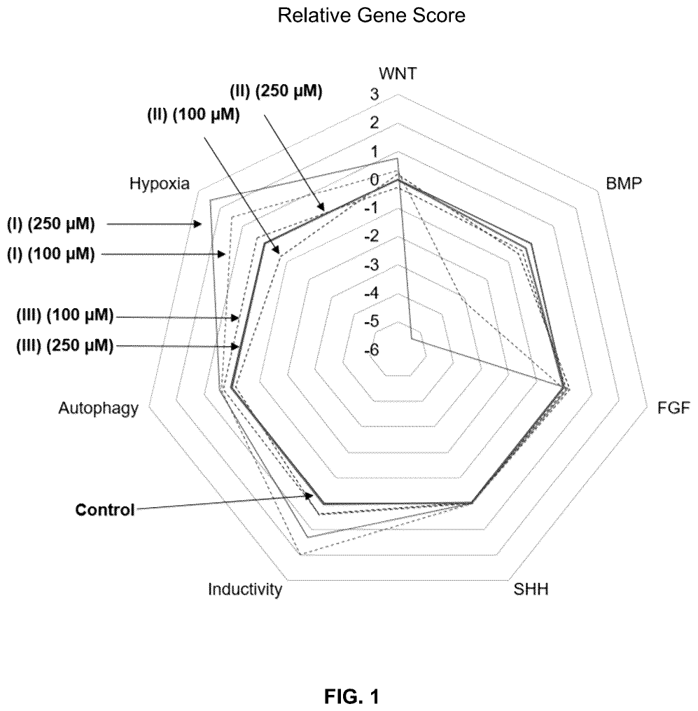

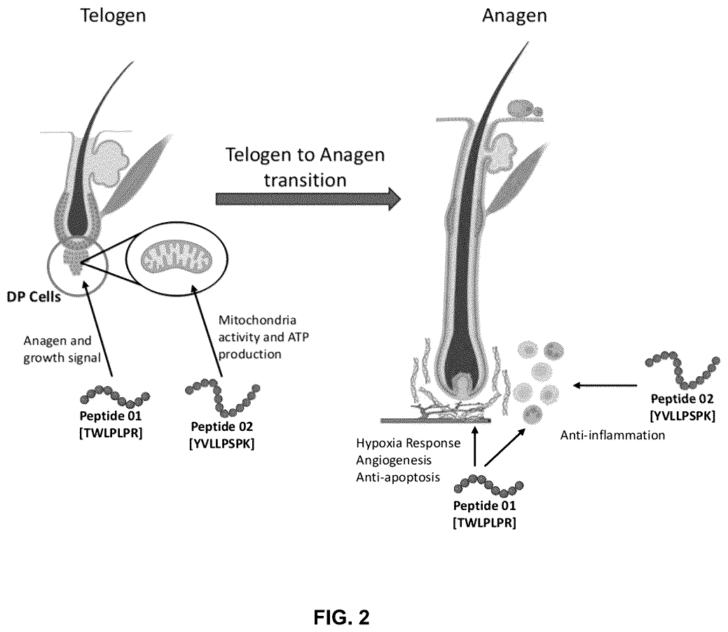

Walnut Peptides (II) and (III) did not have any direct effect on the genes relevant to DPC activity in the context of anagen entry. Therefore, no strong increase of the relative scores over the negative control was observed. The data is illustrated with a circular plot showing relative gene scores in FIG. 1. FIG. 2 shows Walnut Peptide (I) (SEQ ID NO: 45) upregulating growth signals and the anagen phase of follicular hair growth. It also shows Walnut Peptide (II) promoting mitochondrial function and adenosine triphosphate production, thereby helping to meet the energy requirements for active hair growth.

### Example 5

### Example Hair and Scalp Treatment Composition

| INGREDIENTS | | wt. % |
|---|---|---|
| Walnut | PEPTIDE I, II, OR COMBINATIONS | 0.01-10% |
| Peptide(s) | THEREOF | |
| Thickeners | AMMONIUM | 0.8 |
| | POLYACRYLOYLDIMETHYL | |
| | TAURATE | |
| | XANTHAN GUM | 0.2 |
| Preservatives | SODIUM BENZOATE | 0.3 |
| | DISODIUM EDTA | 0.1 |
| | 2-PHENOXYETHANOL | 0.7 |
| Water | WATER | QS 100 |

The foregoing description illustrates and describes preferred embodiments of the inventions. Nonetheless, it is to be understood that the inventions are capable of use in various other combinations, modifications, and environments and are capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the inventions to the forms disclosed herein. Also, it is intended to the appended claims be construed consistent with the specification and to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art. The disclosure may not expressly identify all compounds mentioned throughout the disclosure that may exist as a salt or may be exist in an ionized form, for example, when formulated in a pharmaceutical or cosmetic composition. Nonetheless, to the extent salt or ionized forms of the compound exist and are known, they are intended to be encompassed within the scope of the instant disclosure, even if not expressly mentioned. For example, many surfactants can exist as salts or be in an ionized form in pharmaceutical or cosmetic compositions. Likewise, peptides, include bioactive walnut peptides may be in the form of salts or ionized, which can improve their stability or compatibility. Nonlimiting examples of useful actions include sodium, potassium, calcium, magnesium, chloride, sulfate, phosphate, acetate, citrate, TFA (trifluoroacetate), bicarbonate, and nitrate. Throughout the disclosure, when referencing a bioactive walnut peptide, salts of the bioactive walnut peptides are intended to be covered even though terms such as “a salt thereof” or “salts thereof” is not expressly associated with the peptides or with every instant a peptide is mentioned.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions may be modified in all instances by the term “about,” meaning within +/−5% of the indicated number. For example, an amount of “about 10 wt. %” includes amounts as low as 9.5 wt. % and as high as 10.5 wt. %. An amount of “about 50 wt. %” includes amounts as low as 47.5 wt. % and as high as 52.5 wt. %.

All percentages, parts and ratios herein are based upon weight, unless otherwise indicated.

Some of the various categories of components mentioned throughout the disclosure may overlap. Nonetheless, a single overlapping component cannot simultaneously serve as two different components despite the overlap. Certain alcohols function as both a preservative and as a water-soluble solvent, which may be useful in a physiologically acceptable carrier. If an embodiment, claim, or other recitation throughout the disclosure communicates that both a preservative and a water-soluble solvent are present, needed, or required, a single alcohol such as ethanol cannot simultaneously serve as the preservative and as the water-soluble solvent. In this case, the ethanol shall serve as the preservative or as the water-soluble solvent, but simultaneously as both.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “substantially free” or “essentially free” as used herein up to 2 wt. % of the element may be present. In further embodiments, however, up to 1.5 wt. %, up to 1 wt. %, up to 0.5 wt. %, up to 0.1 wt. %, up to 0.01 wt. % of the element may be present. For example, in the case of a pharmaceutical or cosmetic composition being substantially free or essentially free of a component, the pharmaceutical or cosmetic composition may include up to 2 wt. % of the component. Nonetheless, the pharmaceutical or cosmetic composition preferably includes 1.5 wt. % or less, 1 wt. % or less, 0.5 wt. % or less, 0.1 wt. % or less, 0.01 wt. % or less, or none of the specified component.

As used herein, a “subject” can be a human, non-human primate, mammal, rat, mouse, cow, horse, pig, sheep, goat, dog, cat, and the like. “Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

All components that are positively set forth throughout the disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

1. A method for treating or preventing hair loss, or treating scalp conditions comprising applying to the scalp an effective amount of one or more walnut-derived peptides comprising from 2 to 20 amino acid residues and one or more amino acid sequences selected from:
SEQ ID NO: 1
(Thr-Trp),
SEQ ID NO: 2
(Trp-Leu),
SEQ ID NO: 3
(Leu-Pro),
SEQ ID NO: 4
(Pro-Leu),
SEQ ID NO: 5
(Pro-Arg),
SEQ ID NO: 6
(Tyr-Val),
SEQ ID NO: 7
(Val-Leu),
SEQ ID NO: 8
(Leu-Leu),
SEQ ID NO: 9
(Leu-Pro),
SEQ ID NO: 10
(Pro-Ser),
SEQ ID NO: 11
(Ser-Pro),
and
SEQ ID NO: 12
(Pro-Lys).

2. The method of claim 1, wherein the one or more walnut-derived peptides comprise from 3 to 20 amino acid residues and one or more amino acid sequences selected from:
SEQ ID NO: 13
(Thr-Trp-Leu),
SEQ ID NO: 14
(Trp-Leu-Pro),
SEQ ID NO: 15
(Leu-Pro-Leu),
SEQ ID NO: 16
(Pro-Leu-Pro),
SEQ ID NO: 17
(Leu-Pro-Arg),
SEQ ID NO: 18
(Tyr-Val-Leu),
SEQ ID NO: 19
(Val-Leu-Leu),
SEQ ID NO: 20
(Leu-Leu-Pro),
SEQ ID NO: 21
(Leu-Pro-Ser),
SEQ ID NO: 22
(Pro-Ser-Pro),
and
SEQ ID NO: 23
(Ser-Pro-Lys).

3. The method of claim 1, wherein the one or more walnut-derived peptides comprises from 4 to 20 amino acid residues and one or more amino acid sequences selected from:
SEQ ID NO: 24
(Thr-Trp-Leu-Pro)
SEQ ID NO: 25
(Trp-Leu-Pro-Leu),
SEQ ID NO: 26
(Leu-Pro-Leu-Pro),
SEQ ID NO: 27
(Pro-Leu-Pro-Arg),
SEQ ID NO: 28
(Tyr-Val-Leu-Leu),
SEQ ID NO: 29
(Val-Leu-Leu-Pro),
SEQ ID NO: 30
(Leu-Leu-Pro-Ser),
SEQ ID NO: 31
(Leu-Pro-Ser-Pro),
and
SEQ ID NO: 32
(Pro-Ser-Pro-Lys).

4. The method of claim 1, wherein the one or more walnut-derived peptides comprise from 5 to 18 amino acid residues.

5. The method of claim 4, wherein the one or more walnut-derived peptides comprise one or more amino acid sequences selected from:
SEQ ID NO: 33
(Thr-Trp-Leu-Pro-Leu),
SEQ ID NO: 34
(Trp-Leu-Pro-Leu-Pro),
SEQ ID NO: 35
(Leu-Pro-Leu-Pro-Arg),
SEQ ID NO: 36
(Tyr-Val-Leu-Leu-Pro),
SEQ ID NO: 37
(Val-Leu-Leu-Pro-Ser),
SEQ ID NO: 38
(Leu-Leu-Pro-Ser-Pro),
and
SEQ ID NO: 39
(Leu-Pro-Ser-Pro-Lys).

6. The method of claim 1, wherein the one or more walnut-derived peptides comprise from 6 to 18 amino acid residues.

7. The method of claim 6, wherein the one or more walnut-derived peptides comprise one or more amino acid residues selected from:
SEQ ID NO: 40
(Thr-Trp-Leu-Pro-Leu-Pro),
SEQ ID NO: 41
(Trp-Leu-Pro-Leu-Pro-Arg),
SEQ ID NO: 42
(Tyr-Val-Leu-Leu-Pro-Ser),
SEQ ID NO: 43
(Val-Leu-Leu-Pro-Ser-Pro),
and
SEQ ID NO: 44
(Leu-Leu-Pro-Ser-Pro-Lys).

8. The method of claim 1, wherein the one or more one or more walnut-derived peptides comprise from 4 to 15 amino acid residues, preferably from 5 to 10 amino acid residues.

9. The method of claim 1, wherein the one or more walnut-derived peptides comprise one or more amino acid sequences selected from:
SEQ ID NO: 45
(Thr-Trp-Leu-Pro-Leu-Pro-Arg),
and
SEQ ID NO: 46
(Tyr-Val-Leu-Leu-Pro-Ser-Pro-Lys).

10. The method of claim 9, wherein the one or more walnut-derived peptides are selected from compounds of Formula (I) and Formula (II):
(I)
X-Thr-Trp-Leu-Pro-Leu-Pro-Arg-Z
(II)
X-Tyr-Val-Leu-Leu-Pro-Ser-Pro-Lys-Z,
wherein,
X represents the primary amine function of the N-terminal amino acid, free or substituted by a protecting group selected from an acetyl group, a benzoyl group, a tosyl group, or a benzyloxycarbonyl group, and
Z represents the hydroxyl group of the carboxyl function of the C-terminal amino acid, free or substituted by a protecting group selected from a C₁-C₂₀, NH₂, NHY, or NYY, wherein Y represents a C1-C4 alkyl.

11. The method of claim 1, wherein the one or more walnut-derived peptides correspond to:
SEQ ID NO: 45
(Thr-Trp-Leu-Pro-Leu-Pro-Arg),
and
SEQ ID NO: 46
(Tyr-Val-Leu-Leu-Pro-Ser-Pro-Lys).

12. The method of claim 11, wherein the method comprises applying an effective amount at least two walnut-derived peptides, a first walnut-derived peptide and a second walnut-derived peptide.

13. The method of claim 12, wherein the method comprises applying an effective amount of a walnut-derived peptide comprising the amino acid sequence of SEQ ID NO: 45 and a walnut-derived peptide comprising the amino acid sequence of SEQ ID NO: 46.

14. The method of claim 12, wherein the first walnut-derived peptides and the second walnut-derive peptide are in a weight ratio of about 1:10 to about 10:1.

15. The method of claim 12, wherein the at least two walnut-derived peptides synergistically treats or prevents hair loss.

16. The method of claim 1, wherein the one or more walnut-derived peptides are applied to the hair-bearing skin in a topical composition comprising the one or more walnut-derived peptides and a dermatologically acceptable carrier.

17. The method of claim 15, wherein the topical composition comprises from about 0.001 to about 10 wt. % of the one or more walnut-derived peptides.

18. A topical compositions for treating or preventing hair loss, or treating scalp conditions comprising:
(a) about 0.001 to about 10 wt. % of one or more walnut-derived peptides comprising from 3 to 20 amino acid residues, provided the one or more walnut-derived peptide comprise an amino acid sequences selected from SEQ ID NOS: 1-46;
(b) one or more non-walnut-derived active agents; and
(c) a dermatologically acceptable carrier.

19. The topical composition of claim 18 wherein the one or more non-walnut-derived hair growth promoting agents are selected from androgen receptor inhibitors, androgen antagonists, and an antiandrogens.

20. The topical composition of claim 18, wherein the one or more non-walnut-derived hair growth promoting agents are selected from one or more hair growth promoting agents selected from episteride, finasteride, cyproterone acetate, alfatradiol, minoxidil, bimatoprost, bicalcutamide, spironolactone, flutamide, lantoanoprost, dutasteride, ketoconazole, tofacitinib, ruxolitinib, tacrolimus, bimatoprost, latanoprost, spironolactone, aldactone, kenalog-10, kenalog-40, triamcinolone, azulfidine, sulfasalazine, and sulfazine.


SEQUENCE LISTING (47 sequences)

SEQ ID NO: 1-23 - skipped (23 entries)

SEQ ID NO: 24 - protein, 4 aa, synthetic construct
TWLP

SEQ ID NO: 25 - protein, 4 aa, synthetic construct
WLPL

SEQ ID NO: 26 - protein, 4 aa, synthetic construct
LPLP

SEQ ID NO: 27 - protein, 4 aa, synthetic construct
PLPR

SEQ ID NO: 28 - protein, 4 aa, synthetic construct
YVLL

SEQ ID NO: 29 - protein, 4 aa, synthetic construct
VLLP

SEQ ID NO: 30 - protein, 4 aa, synthetic construct
LLPS

SEQ ID NO: 31 - protein, 4 aa, synthetic construct
LPSP

SEQ ID NO: 32 - protein, 4 aa, synthetic construct
PSPK

SEQ ID NO: 33 - protein, 5 aa, synthetic construct
TWLPL

SEQ ID NO: 34 - protein, 5 aa, synthetic construct
WLPLP

SEQ ID NO: 35 - protein, 5 aa, synthetic construct
LPLPR

SEQ ID NO: 36 - protein, 5 aa, synthetic construct
YVLLP

SEQ ID NO: 37 - protein, 5 aa, synthetic construct
VLLPS

SEQ ID NO: 38 - protein, 5 aa, synthetic construct
LLPSP

SEQ ID NO: 39 - protein, 5 aa, synthetic construct
LPSPK

SEQ ID NO: 40 - protein, 6 aa, synthetic construct
TWLPLP

SEQ ID NO: 41 - protein, 6 aa, synthetic construct
WLPLPR

SEQ ID NO: 42 - protein, 6 aa, synthetic construct
YVLLPS

SEQ ID NO: 43 - protein, 6 aa, synthetic construct
VLLPSP

SEQ ID NO: 44 - protein, 6 aa, synthetic construct
LLPSPK

SEQ ID NO: 45 - protein, 7 aa, synthetic construct
TWLPLPR

SEQ ID NO: 46 - protein, 8 aa, synthetic construct
YVLLPSPK

SEQ ID NO: 47 - protein, 7 aa, synthetic construct
KVPPLLY